(19) United States
(12) Patent Application Publication
(10) Pub. No.: US 2012/0135037 A1
(43) Pub. Date: May 31, 2012

(54) FLAGELLIN FUSION PROTEINS AND CONJUGATES COMPRISING PNEUMOCOCCUS ANTIGENS AND METHODS OF USING THE SAME

(76) Inventors: Steven B. Mizel, Lewisville, NC (US); Sean Reid, High Point, NC (US)

(21) Appl. No.: 13/375,417

(22) PCT Filed: May 27, 2010
(86) PCT No.: PCT/US2010/036369

§ 371 Date: Feb. 13, 2012

Related U.S. Application Data
(60) Provisional application No. 61/182,978, filed on Jun. 1, 2009.

(51) Int. Cl. A61K 39/09; C12N 5/10; A61P 37/04; C12N 1/21; A01N 37/18; C12N 15/63; A61K 38/16; C07K 14/00; A61P 31/04; A61J 1/00; C12P 21/06; C12N 1/19

(52) U.S. Cl. 424/244.1; 435/69.7; 435/325; 435/348; 435/350; 435/351; 435/352; 435/353; 435/354; 435/366; 435/419; 435/252.3; 435/252.33; 435/254.2; 435/320.1; 514/1.1; 530/350; 530/395; 536/23.4; 206/438; 514/2.6

(57) ABSTRACT

The present invention is based, in part, on flagellin adjuvant used to enhance immune responses directed against Streptococcus pneumoniae, in particular, to enhance immune responses to polypeptide antigens (e.g., PspA) and capsular polysaccharide from S. pneumoniae. In representative embodiments, the invention provides a fusion protein comprising a flagellin adjuvant and one more polypeptide antigens from S. pneumoniae. In other embodiments, the invention provides a conjugate comprising a flagellin adjuvant covalently linked to a capsular polysaccharide from one or more serotypes of S. pneumoniae. Also provided are compositions comprising the fusion proteins and/or conjugates of the invention as well as immunogenic formulations comprising the inventive fusion proteins, conjugates and/or compositions. The invention also provides methods of producing an immune response against S. pneumoniae and methods of protecting a subject from S. pneumoniae infection by administering the fusion proteins, conjugates, compositions and/or immunogenic formulations of the invention to the subject.

### RELATED APPLICATION INFORMATION

This application claims the benefit of U.S. Provisional Application No. 61/182,978; Filed Jun. 1, 2009, the disclosure of which is incorporated by reference herein in its entirety.

### STATEMENT OF FEDERAL SUPPORT

This invention was made, in part, with government support under grant number AI070440 from the National Institute of Allergy and Infectious Diseases of the National Institutes of Health. The United States government has certain rights to this invention.

### FIELD OF THE INVENTION

The present invention relates to immunogenic compositions comprising antigens from Streptococcus pneumoniae and methods of using the same to induce an immune response in a subject.

### BACKGROUND OF THE INVENTION

Streptococcus pneumoniae (also known as pneumococcus) is a Gram-positive pathogenic bacterium, and a significant source of human morbidity and mortality. There are approximately 90 known serotypes, with particular serotypes being more commonly associated with disease. Pneumococcus infection can result in pneumonia meningitis, acute sinusitis, otitis media, bacteremia, osteomyelitis, septic arthritis, endocarditis, peritonitis, pericarditis, cellulitis and brain abscesses. In particular, pneumococcus infection is the most common cause of bacterial meningitis in both adults and children.

There is an increasing incidence of drug-resistant strains of pneumococcus, underscoring the need for effective vaccine strategies. Current approaches include immunization with capsular polysaccharides from multiple serotypes of pneumococcus conjugated to a carrier. For example, PREVNAR® and PREVNAR 13® (produced by Wyeth) comprise capsular polysaccharides from 7 or 13 serotypes of pneumococcus, respectively, chemically conjugated to a diphtheria toxoid (CRM₁₉₇). SYNFLORIX® is produced by GlaxoSmithKline and is a 10-valent vaccine comprising capsular polysaccharides from 10 serotypes of pneumococcus conjugated to a Haemophilus influenzae protein D. Merck & Co. markets a pneumococcal vaccine, PNEUMOVAX® 23, which contains unconjugated capsular polysaccharides from 23 pneumococcal serotypes.

Clinically, conjugated vaccines have primarily been targeted at infants and toddlers, whereas non-conjugated vaccines have generally been indicated for subjects over two years of age.

### SUMMARY OF THE INVENTION

The present invention is based, in part, on flagellin adjuvants that enhance immune responses directed against Streptococcus pneumoniae, in particular, to enhance immune responses to polypeptide antigens (e.g., PspA) and capsular polysaccharide from S. pneumoniae. In representative embodiments, the invention provides a fusion protein comprising a flagellin adjuvant and one more polypeptide antigens from S. pneumoniae. In other embodiments, the invention provides a conjugate comprising a flagellin adjuvant covalently linked to a capsular polysaccharide from one or more serotypes of S. pneumoniae. Also provided are compositions comprising the fusion proteins and/or conjugates of the invention as well as immunogenic formulations comprising the inventive fusion proteins, conjugates and/or compositions. The fusion proteins, conjugates, compositions and immunogenic formulations can be administered to a subject, e.g., to induce an immune response against S. pneumoniae or as a method of protecting a subject from S. pneumoniae infection.

Because of the strong adjuvant effect of flagellin, in embodiments of the invention administration of the flagellin fusion proteins, conjugates, compositions and/or formulations to a subject (e.g., mammalian subject) induces strong humoral (e.g., circulating IgG) and/or cellular (e.g., T cell) responses that generate immunological memory (e.g., B memory cells). Further, in embodiments of the invention, protective immune responses can be achieved with relatively low dosages of the flagellin fusion protein, conjugate, composition and/or immunogenic formulation.

Thus, in representative embodiments, the fusion proteins, conjugates, compositions and/or formulations of the invention may produce enhanced immune responses in a subject as compared with existing S. pneumoniae vaccines, for example, enhanced antibody titers and/or quality of the antibody response (for example, in terms of IgG isotype and/or affinity of the antibodies for antigen) and/or a requirement for lower dosages to achieve protection against S. pneumoniae. In other representative embodiments, the fusion proteins, conjugates, compositions and/or formulations of the invention may provide longer term protection as compared with existing vaccines and/or may be safer and/or have fewer side effects and/or require fewer dosages to achieve an immune response and/or protection. In particular embodiments, these advantageous effects may be achieved in particular patient populations, e.g., in infants and toddlers (e.g., children under about two years of age), older patients that have not previously been vaccinated against S. pneumoniae and/or older patient populations at heightened risk for S. pneumoniae infection.

Accordingly, as one aspect, the invention provides a fusion protein comprising a flagellin adjuvant and a S. pneumoniae PspA antigen. Also provided are nucleic acids and vectors encoding the fusion protein, and host cells comprising the nucleic acids or vectors.

As a further aspect, the invention provides a method of making a fusion protein of the invention, the method comprising culturing a host cell of the invention in a culture medium under conditions sufficient for the fusion protein to be produced.

As still another aspect, the invention provides a conjugate comprising a flagellin adjuvant covalently linked to capsular polysaccharide from one or more serotypes of S. pneumoniae.

As yet another aspect, the invention provides a composition comprising:
(a) a fusion protein of the invention; and
(b) capsular polysaccharide from one or more serotypes of S. pneumoniae.

Also provided is a composition comprising:
(a) a fusion protein of the invention; and
(b) a conjugate of the invention.

Still further, the invention provides a composition comprising:
(a) two or more fusion proteins of the invention; and/or
(b) two or more conjugates of the invention.

As a further aspect, the invention provides an immunogenic formulation comprising a fusion protein, conjugate and/or composition of the invention in a pharmaceutically acceptable carrier.

As another aspect, the invention provides a method of producing an immune response against S. pneumoniae in a subject (e.g., a mammalian subject), the method comprising administering a fusion protein, conjugate, composition and/or immunogenic formulation of the invention to the subject in an amount effective to produce an immune response in the subject against S. pneumoniae.

Still further, the invention provides a method of protecting a subject (e.g., mammalian subject) from infection with S. pneumoniae, the method comprising administering a fusion protein, conjugate, composition and/or immunogenic formulation of the invention to the subject in an amount effective to protect the subject from infection with S. pneumoniae.

As yet another aspect, the invention provides a method of enhancing a protective immune response to S. pneumoniae in a subject (e.g., mammalian subject), the method comprising administering a fusion protein, conjugate, composition and/or immunogenic formulation of the invention to the subject in an amount effective to enhance the protective immune response to S. pneumoniae in the subject.

In representative embodiments, the methods of the invention are carried out with a child of less than about two or less than about five years of age. In representative embodiments of the invention wherein the subject is a child of less than about two years of age administered capsular polysaccharide from one or more serotypes of S. pneumoniae, the capsular polysaccharide is generally a conjugated polysaccharide.

The invention also encompasses an article of manufacture comprising a closed, pathogen-impermeable container and a sterile vaccine preparation enclosed within the container, wherein the vaccine preparation comprises an immunogenic formulation of the invention.

The invention also provides for the use of a flagellin fusion protein, flagellin conjugate, composition and/or immunogenic formulation of the invention for the manufacture of a medicament for inducing an immune response (optionally, a protective immune response) against S. pneumoniae in a subject, for protecting a subject from infection with S. pneumoniae, and/or for enhancing a protective immune response to S. pneumoniae in a subject. In embodiments, the subject is a mammalian subject, optionally a child less than about two or five years of age.

Also provided is a flagellin fusion protein, flagellin conjugate, composition and/or immunogenic formulation of the invention for inducing an immune response (optionally, a protective immune response) against S. pneumoniae in a subject, for protecting a subject from infection with S. pneumoniae, and/or for enhancing a protective immune response to S. pneumoniae in a subject. In embodiments, the subject is a mammalian subject, optionally a child less than about two or five years of age.

These and other aspects of the invention are set forth in more detail in the description of the invention below.

### BRIEF DESCRIPTION OF THE DRAWINGS

FIG. 1 shows plasma titers of anti-PspA IgG (determined by ELISA) in mice immunized with 1.05 micrograms of PspA, 3.5 micrograms of PspA, 3 micrograms PspA-flagellin fusion protein, or 10 micrograms of PspA-flagellin fusion protein.

### DETAILED DESCRIPTION OF THE INVENTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. §1.822 and established usage.

### I. DEFINITIONS

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

Numerical ranges as described herein are intended to be inclusive unless the context indicates otherwise. For example, the numerical range of “1 to 10” or “1-10” is intended to be inclusive of the values 1 and 10.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

The term “about,” as used herein when referring to a measurable value such as an amount of polypeptide, dose, time, temperature, enzymatic activity or other biological activity and the like, is meant to encompass variations of ±20%, ±10%, ±5%, ±1%, ±0.5%, or even ±0.1% of the specified amount.

By “consisting essentially of” (and grammatical variants) as used herein, it is meant that the indicated polypeptide, conjugate, nucleic acid, composition, formulation and the like does not include any other material elements (i.e., elements that materially impact the structure and/or function of the polypeptide, conjugate, nucleic acid, composition or formulation). The term “materially altered,” as applied to nucleic acids of the invention, refers to an increase or decrease in the ability to express the encoded polypeptide of at least about 50% or more as compared with the expression level of a nucleic acid consisting of the recited sequence. The term “materially altered,” as applied to polypeptides, conjugates, compositions and immunogenic formulations of the invention, refers to an increase or decrease in immunogenic or adjuvant activity of at least about 25% or 50% or more as compared with the activity of a polypeptide, conjugate, composition or immunogenic formulation consisting of the recited elements. In representative embodiments, the term “consisting essentially of” (and grammatical variants), as applied to a nucleic acid or polypeptide sequence of this invention, means a nucleic acid or polypeptide that consists of both the recited sequence (e.g., SEQ ID NO) and a total of 10 or less (e.g., 1, 2, 3, 4, 5, 6, 7, 8, 9, or 10) additional nucleotides or amino acids on the 5′ and/or 3′ or N-terminal and/or C-terminal ends of the recited sequence such that the function of the polynucleotide or polypeptide is not materially altered. The total of ten or less additional nucleotides or amino acids includes the total number of additional nucleotides or amino acids on both ends added together. Thus, as used herein, the term “consisting essentially of” is not to be interpreted as meaning “comprising.”

As used herein, the term “polypeptide” encompasses both peptides and proteins (including fusion proteins), unless indicated otherwise.

A “fusion protein” is a polypeptide produced when two heterologous nucleotide sequences or fragments thereof coding for two (or more) different polypeptides not found fused together in nature are fused together in the correct translational reading frame.

The terms “immunogen” and “antigen” are used interchangeably herein and mean any compound (including polypeptides, polysaccharides, etc.) to which a cellular and/or humoral immune response can be directed.

As used herein, the terms “enhance,” “enhances,” and “enhancing” an immune response (and similar terms), optionally a protective immune response, indicate that the immune response (e.g., antigen-specific IgG production), optionally a protective immune response, is increased by at least about 50%, 2-fold, 3-fold, 4-fold, 5-fold, 10-fold, 15-fold, 20-fold, 30-fold, 40-fold, 50-fold, 60-fold, 75-fold, 100-fold, 150-fold, 500-fold, 1000-fold or more.

As used herein, an amino acid sequence that is “substantially identical” or “substantially similar” to a reference amino acid sequence is at least about 75%, 80%, 85%, 90%, 95%, 97%, 98% or 99% identical or similar, respectively, to the reference amino acid sequence.

Methods of determining sequence similarity or identity between two or more amino acid sequences are known in the art. Sequence similarity or identity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J. Mol. Biol. 48,443 (1970), by the search for similarity method of Pearson & Lipman, Proc. Natl. Acad. Sci. USA 85,2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12, 387-395 (1984), or by inspection.

Another suitable algorithm is the BLAST algorithm, described in Altschul et al., J. Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); http://blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity.

Further, an additional useful algorithm is gapped BLAST as reported by Altschul et al., (1997) Nucleic Acids Res. 25, 3389-3402.

In representative embodiments of the invention, the polypeptides, capsular polysaccharide, nucleic acids and/or cells of the invention are “isolated.” By “isolated” it is meant that the polypeptide, capsular polysaccharide, nucleic acid and/or cell is at least partially purified away from some of the other components of the naturally occurring organism or virus with which it is naturally associated. To illustrate, unless the context indicates otherwise, the isolated fusion proteins of the invention are generally not incorporated into flagella, either as part of an organism or as isolated flagella. In representative embodiments of the invention an “isolated” peptide, protein or fusion protein is at least about 5%, 10%, 15%, 20%, 25%, 30%, 40%, 50%, 60%, 70%, 75%, 80%, 85%, 90%, 95%, 97%, 98%, 99% pure (w/w) or more.

By the term “treat,” “treating” or “treatment of” (and grammatical variations thereof) it is meant that the severity of the subject's condition is reduced, at least partially improved or ameliorated and/or that some alleviation, mitigation or decrease in at least one clinical symptom is achieved and/or there is a delay in the progression of the disease or disorder.

A “treatment effective” amount as used herein is an amount that is sufficient to treat (as defined herein) the subject. Those skilled in the art will appreciate that the therapeutic effects need not be complete or curative, as long as some benefit is provided to the subject.

The terms “prevent,” “preventing” and “prevention of” (and grammatical variations thereof) refer to prevention and/or delay of the onset and/or progression of a disease, disorder and/or a clinical symptom(s) in a subject and/or a reduction in the severity of the onset and/or progression of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset and/or the progression is less than what would occur in the absence of the present invention.

A “prevention effective” amount as used herein is an amount that is sufficient to prevent (as defined herein) the disease, disorder and/or clinical symptom in the subject. Those skilled in the art will appreciate that the level of prevention need not be complete, as long as some benefit is provided to the subject.

The terms “vaccination” or “immunization” are well-understood in the art, and are used interchangeably herein unless otherwise indicated. For example, the terms vaccination or immunization can be understood to be a process that increases an organism's immune response to antigen and therefore to resist or overcome infection. In the case of the present invention, vaccination or immunization against S. pneumoniae increases the organism's immune response to resist or overcome infection by S. pneumoniae.

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to immunogens by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

The terms “protective” immune response or “protective” immunity as used herein indicates that the immune response confers some benefit to the subject in that it prevents or reduces the incidence and/or severity and/or duration of disease. Alternatively, a protective immune response or protective immunity may be useful in the therapeutic treatment of existing disease.

Unless indicated otherwise, the terms “protect,” “protecting,” “protection” and “protective” (and grammatical variations thereof) encompass both methods of preventing and treating S. pneumoniae infection in a subject.

### II. STREPTOCOCCUS PNEUMONIAE ANTIGENS

The present invention can be practiced with any suitable S. pneumoniae antigen including, without limitation, polypeptide antigens (including glyco-polypeptides and lipo-polypeptides), polysaccharide antigens (including lipopolysaccharides), and nucleic acid antigens.

A. Polypeptide Antigens. Streptococcus pneumoniae polypeptide antigens according to the present invention encompass any suitable S. pneumoniae polypeptide antigen. In representative embodiments, the S. pneumoniae polypeptide antigen is an antigen derived from a S. pneumoniae protein that is exposed on the outer surface of the organism and/or is secreted and/or released by the organism. Nonlimiting examples of S. pneumoniae polypeptide antigens include antigens derived from a S. pneumoniae toxin, adhesin, signal transducer and/or lipoprotein (including the entire polypeptide and active fragments thereof). Further examples include without limitation, one or more of a S. pneumoniae PspA antigen, a S. pneumoniae PsaA antigen, a S. pneumoniae PspC antigen (optionally, a transmembrane deletion form), a S. pneumoniae PcpA antigen, a S. pneumoniae PdB antigen, a S. pneumoniae histidine triad protein antigen (PhtA, PhtB, PhtD and/or PhtE), a S. pneumoniae pilus protein subunit antigen, a S. pneumoniae pneumolysin antigen (optionally a detoxified form), a S. pneumoniae PiuA antigen, a S. pneumoniae PiaA antigen, a S. pneumoniae choline binding protein antigen (optionally, a transmembrane deletion form), a S. pneumoniae CbpA antigen (optionally, a transmembrane deletion form), a S. pneumoniae glyceraldehyde-3-phospate dehydrogenase antigen, a S. pneumoniae M like protein antigen, a S. pneumoniae HSP70 antigen, and a S. pneumoniae Usp45 antigen (these terms including the entire polypeptide and active fragments thereof); or fusions of two or more S. pneumoniae antigens (these terms including the entire polypeptide and active fragments thereof). In particular embodiments, the PspA antigen is a PspA1 antigen, a PspA2 antigen, a PspA3 antigen, a PspA4 antigen, a PspA5 antigen and/or a PspA6 antigen (these terms including the entire polypeptide and active fragments thereof) or a PspA antigen from any other PspA clade now known or later identified; or fusions of two or more of the foregoing. Suitable active fragments generally comprise one or more epitopes that induce an immune response (cellular and/or humoral) and, optionally, confer protection to a subject against S. pneumoniae. In representative embodiments, the active fragment comprises all or part of an extracellular portion of the polypeptide.

In embodiments of the invention, an “active fragment” of a S. pneumoniae polypeptide antigen or epitope is at least about 6, 8, 10, 15, 20, 30, 50, 75, 100, 150, 200, 250 or 300 or more contiguous amino acids and/or less than about 300, 250, 200, 150, 100, 75, 50, 30, 20 or 15 contiguous amino acids, including any combination of the foregoing as long as the lower limit is less than the upper limit and induces an immune response (e.g., IgG that react with the native antigen), optionally a protective immune response, against S. pneumoniae in a host. In particular embodiments, the active fragment induces an immune response in a host, optionally a protective immune response, that is at least about 50%, 75%, 80%, 85%, 90%, or 95% or more of the immune response induced by the full-length antigen or epitope, or induces an immune response that is the same as or essentially the same as the full-length antigen or epitope, or induces an immune response that is even greater than the immune response induced by the full-length antigen or epitope.

Further, as used herein, a “S. pneumoniae antigen” or “antigen from S. pneumoniae” or like terms include, without limitation, naturally occurring S. pneumoniae antigens and modified forms thereof that induce an immune response in a subject, optionally a protective immune response, against S. pneumoniae. For example, a native polypeptide antigen can be modified to increase safety and/or immunogenicity and/or as a result of cloning procedures or other laboratory manipulations. Further, in embodiments of the invention, the amino acid sequence of the modified form of the S. pneumoniae polypeptide antigen can comprise one, two, three or fewer, four or fewer, five or fewer, six or fewer, seven or fewer, eight or fewer, nine or fewer, or ten or fewer modifications as compared with the amino acid sequence of the naturally occurring antigen and induce an immune response (optionally a protective immune response) against S. pneumoniae in the host. Suitable modifications encompass deletions (including truncations), insertions (including N- and/or C-terminal extensions) and amino acid substitutions, and any combination thereof. In representative embodiments, the S. pneumoniae polypeptide antigen is a polypeptide antigen that is substantially similar at the amino acid level to the amino acid sequence of a naturally occurring S. pneumoniae polypeptide antigen and induces an immune response (optionally a protective immune response) against S. pneumoniae in a host.

In embodiments of the invention, a “modified” S. pneumoniae antigen or epitope induces an immune response in a host (e.g., IgG that react with the native antigen), optionally a protective immune response, that is at least about 50%, 75%, 80%, 85%, 90%, or 95% or more of the immune response induced by the native antigen or epitope, or induces an immune response that is the same as or essentially the same as the native antigen or epitope, or induces an immune response that is even greater than the immune response induced by the native antigen or epitope.

In embodiments of the invention, two or more S. pneumoniae antigens are provided in the fusion protein and/or composition (e.g., 2, 3, 4, 5, 6 or more S. pneumoniae antigens), for example, one or more of a S. pneumoniae PspA antigen, a S. pneumoniae PsaA antigen, a S. pneumoniae PspC antigen (optionally, a transmembrane deletion form), a S. pneumoniae PcpA antigen, a S. pneumoniae PdB antigen, a S. pneumoniae histidine triad protein antigen (PhtA, PhtB, PhtD and/or PhtE), a S. pneumoniae pilus protein subunit antigen, a S. pneumoniae pneumolysin antigen (optionally a detoxified form), a S. pneumoniae PiuA antigen, a S. pneumoniae PiaA antigen, a S. pneumoniae choline binding protein antigen (optionally, a transmembrane deletion form), a S. pneumoniae CbpA antigen (optionally, a transmembrane deletion form), a S. pneumoniae glyceraldehyde-3-phospate dehydrogenase antigen, a S. pneumoniae M like protein antigen, a S. pneumoniae HSP70 antigen, and a S. pneumoniae Usp45 antigen (these terms including the entire polypeptide and active fragments thereof). For example, a S. pneumoniae PspA antigen and a S. pneumoniae PsaA antigen can be provided. As a further exemplary embodiment, a S. pneumoniae PiuA and a S. pneumoniae PiaA antigen can be provided. Further, fusion proteins and/or compositions of the invention can comprise two or more (e.g., two, three, four, five or six) of a S. pneumoniae PspA1 antigen, a S. pneumoniae PspA2 antigen, a S. pneumoniae PspA3 antigen, a S. pneumoniae PspA4 antigen, a S. pneumoniae PspA5 antigen, and a S. pneumoniae PspA6 antigen.

Further, in embodiments of the invention, one or more S. pneumoniae antigens can be present in multiple copies (e.g., 2, 3, 4, 5, 6 or more) in the fusion protein and/or composition. For example, a single flagellin fusion protein can comprise two or more copies of a S. pneumoniae antigen and/or in a composition comprising multiple flagellin fusion proteins of the invention, a S. pneumoniae antigen can be present in two or more of the fusion proteins in the composition. As a further option, a S. pneumoniae antigen can be present in a composition, but not as part of a flagellin fusion protein.

Those skilled in the art will appreciate that it may be advantageous for the antigen to include one or more B cell epitopes and/or one or more T cell epitopes (e.g., 2, 3, 4, 5, 6, 7, 8, 9, 10 or more B cell epitopes and/or T cell epitopes). Optionally, the S. pneumoniae antigen comprises one or more epitopes exposed on the surface of the naturally occurring S. pneumoniae protein.

The S. pneumoniae PspA1, PspA2, PspA3 and PspA4 proteins have been cloned and sequenced (see, Hollingshead et al. (2000) Infect. Immun. 68:5889-5900 and Table 1).

PspA proteins have been well-characterized, including epitope mapping studies (see, e.g., Roche et al., (2003) Infect. Immunity 71:1033-1041); U.S. Pat. No. 5,804,193; U.S. Pat. No. 5,679,768; U.S. Pat. No. 5,965,141; U.S. Pat. No. 5,980,909; and U.S. Pat. No. 5,997,882). PspA has three major protein domains. The amino terminal portion comprises an alpha-helical domain and is exposed on the cell surface. This region is followed by a proline-rich domain which spans the cell wall and capsule layer. The carboxy-terminal portion comprises a choline-binding domain, which attaches the PspA protein to the cell surface.

TABLE 1
Accession Nos. for Nucleic Acid and Amino
Acid Sequences for PspA Clades 1 to 6.

| Clade | Accession No. |
|---|---|
| PspA1 | GENBANK Accession Nos. AF071804 (strain BG9739), |
| (Clade 1) | AF071805 (strain DBL6A), AF071809 (strain L81905), |
| | AF071803 (strain BG8743), AF071802 (strain AC94), |
| | AF071808 (strain BG6692), AF071807 (strain BG8838), |
| | and AF071806 (strain DBL1) |
| PspA2 | GENBANK Accession Nos. M74122 (strain Rx1), |
| (Clade 2) | AF071811 (strain E134), AF071812 (strain EF10197), |
| | AF071813 (strain EF6796), AF071815 (strain BG9163), |
| | AF071810 (strain DBL5), and AF071814 (strain WU2) |
| PspA3 | GENBANK Accession Nos. AF071816 (strain EF3296), |
| (Clade 3) | AF071817 (strain BG8090), and AF071818 (strain |
| | AC122) |
| PspA4 | GENBANK Accession Nos. U89711 (strain EF5668), |
| (Clade 4) | AF071824 (strain BG7561), AF071826 (strain BG7817), |
| | and AF071821 (strain BG11703) |
| PspA5 | GENBANK Accession No. AF071820 (strain ATCC6303) |
| (Clade 5) | |
| PspA6 | GENBANK Accession No. AF071823 (strain BG6380) |
| (Clade 6) | |

U.S. Pat. No. 5,997,882 discloses regions of the PspA protein from the Rx1 strain of S. pneumoniae that contain protection-eliciting epitopes that are cross-reactive with PspAs of other S. pneumoniae strains. One region comprises the 68-amino acid sequence extending from amino acid residues 192 to 260 of the Rx1 strain PspA (clade 2), while another region comprises the C-terminal amino acid sequence extending from amino acid residues 293 to 588 of the PspA protein (Rx1 strain).

U.S. Pat. No. 5,679,768 describes C-terminal truncated PspA proteins (strain Rx1) that lack the cell membrane anchor regions.

U.S. Patent Publication 20020102242 to Briles et al. describes epitopic regions of PspA (strain Rx1) including amino acids 1 to 115, amino acids 1 to 314, amino acids 192 to 260, and amino acids 192 to 588.

Roche et al. ((2003) Infect. Immun. 71(3): 1033-1041) indicate that the fragments containing amino acids 314 to 418 of PspA3 from strain EF3296 are able to elicit cross-protection against pneumococci expressing PspA proteins of clades 2, 3, 4, and 5. An amino acid 1 to 115 fragment (N-terminal alpha-helical region) elicited some cross-protection against clades 2 and 4 in BALB/c mice but not in CBA/N mice.

Roche et al. ((2003) Infect. Immun. 71(8): 4498-4505) used PspA amino acids 1 to 478 (strain EF3296), amino acids 314 to 418 (strain EF3296), and amino acids 170 to 288 (strain Rx1) to immunize mice and provide protection against pneumococci.

In particular embodiments, the S. pneumoniae PspA antigen comprises, consists essentially of, or consists of one or more, two or more, three or more, or four or more epitopes, which may include one or more of the epitopes specifically described herein or homologues or active fragments thereof. In embodiments of the invention, a S. pneumoniae PspA antigen comprises, consists essentially of, or consists of the full-length precursor or mature S. pneumoniae PspA protein or, alternatively, an active fragment of either of the foregoing (e.g., at least about 6, 8, 10, 15, 20, 30, 50, 75, 100, 150, 200, 250, 300 or more contiguous amino acids and/or less than about 300, 250, 200, 150, 100, 75, 50, 30, 20, or 15 contiguous amino acids, including any combination of the foregoing as long as the lower limit is less than the upper limit). In embodiments of the invention, the S. pneumoniae PspA antigen comprises, consists essentially, or consists of the N-terminal alpha-helical region, the proline-rich domain, and/or the C-terminal choline-binding domain or active fragments thereof.

Multiple S. pneumoniae antigens may be provided in the form of a fusion peptide, such as a fusion peptide comprising two or more (e.g., two, three, four, five or six) of a S. pneumoniae PspA1 antigen, a S. pneumoniae PspA2 antigen, a S. pneumoniae PspA3 antigen, a S. pneumoniae PspA4 antigen, a S. pneumoniae PspA5 antigen and/or a S. pneumoniae PspA6 antigen. Where two antigens are joined as a fusion peptide, they may be joined directly to one another or joined by an intervening amino acid sequence such as a peptide linking or “hinge” segment (e.g., a segment of 1, 2, 3, 4, 6, 8, 10, 15, 20, 30, 50 or more amino acids) and/or another antigen, but generally without any intervening flagellin sequences.

Other suitable S. pneumoniae antigens (e.g., PspA antigens), including fusion peptides thereof, in addition to the antigens specifically disclosed herein can be readily identified by those skilled in the art without departing from the present invention.

### B. Capsular Polysaccharides.

Another aspect of the invention is directed to conjugates comprising a flagellin adjuvant (described in more detail herein) covalently linked to one or more S. pneumoniae antigens. In embodiments of the invention, the term “covalently linked” is not intended to refer to a peptide bond (e.g., a fusion protein) between the flagellin adjuvant and the one or more S. pneumoniae antigen. In embodiments of the invention, the term “covalently lined” encompasses peptide bonds. Optionally, the covalent linkage is a direct covalent linkage and/or comprises cross-linking between the flagellin adjuvant and the one or more S. pneumoniae antigens. In representative embodiments, the covalent linkage comprises a linker between the flagellin adjuvant and the S. pneumoniae antigen. The S. pneumoniae antigen can be any suitable antigen including without limitation polypeptides (including glyco-polypeptides and lipo-polypeptides), polysaccharides (including glycol-polysaccharides and lipo-polysaccharides), and nucleic acids.

In embodiments of the invention, the S. pneumoniae antigen is a capsular polysaccharide. There are at least 90 capsular serotypes of S. pneumoniae, which are further classified into 46 serogroups based on the structural and chemical composition of the capsular polysaccharides (Barocchi et al., (2007) Vaccine 25:2963-2973). In representative embodiments, the invention provides a conjugate comprising a flagellin adjuvant covalently linked to a capsular polysaccharide from one or more serotypes of S. pneumoniae (e.g., 2, 3, 4, 5, 6, 7, 8, 9, 10, 11, 12, 13, 15, 23, 25, 28, 30 or more serotypes of S. pneumoniae). The capsular polysaccharide can be from any one or more serotypes of S. pneumoniae now known or later identified. All S. pneumoniae serotypes are designated herein using the Danish nomenclature.

In embodiments of the invention, the capsular polysaccharide comprises, consists essentially of, or consists of capsular polysaccharide from one or more of S. pneumoniae serotypes 4, 6B, 9V, 14, 18C, 19F and/or 23F.

In further embodiments of the invention, the capsular polysaccharide comprises, consists essentially of, or consists of capsular polysaccharide from one or more of S. pneumoniae serotypes 1, 3, 4, 5, 6A, 6B, 7F, 9V, 14, 18C, 19A, 19F and/or 23F.

In representative embodiments of the invention, the capsular polysaccharide comprises, consists essentially of, or consists of capsular polysaccharide from one or more of S. pneumoniae serotypes 1, 4, 5, 6B, 7F, 9V, 14, 18C, 19F and/or 23F.

In embodiments of the invention, the capsular polysaccharide comprises, consists essentially of, or consists of capsular polysaccharide from one or more of S. pneumoniae serotypes 1, 2, 3, 4, 5, 6B, 7F, 8, 9N, 9V, 10A, 11A, 12F, 14, 15B, 17F, 18C, 19A, 19F, 20, 22F, 23F and/or 33F.

In embodiments of the invention, the capsular polysaccharide comprises, consists essentially of, or consists of capsular polysaccharide from one or more of S. pneumoniae serotypes 1, 3, 4, 5, 6B, 7F, 9V, 14, 18C, 19F and/or 23F.

In embodiments of the invention, the capsular polysaccharide comprises, consists essentially of, or consists of capsular polysaccharide from one or more serotypes of S. pneumoniae that are more commonly associated with drug-resistant infections (e.g., penicillin resistant infections, erythromycin resistant infections, trimethoprim-sulfamethoxazole resistant infections and/or extended-spectrum cephalosporin resistant infections), for example, capsular polysaccharide from one or more of S. pneumoniae serotypes 6B, 9V, 14, 19A, 19F and/or 23F.

In further representative embodiments, the capsular polysaccharide comprises, consists essentially of, or consists of capsular polysaccharide from one or more serotypes of S. pneumoniae that are more prevalent in infections seen in developing countries, for example, capsular polysaccharide from one or more of S. pneumoniae serotypes 1, 3, 5 and/or 7F.

Serotype 19A is becoming an increasing public health concern. In particular embodiments, the capsular polysaccharide comprises, consists essentially of, or consists of capsular polysaccharide from serotype 19A.

In representative embodiments, the capsular polysaccharide comprises, consists essentially of, or consists of capsular polysaccharide from serotype 6A and/or serotype 22F.

Compositions comprising pneumococcal capsular polysaccharides (conjugated or unconjugated) are well-known in the art (e.g., U.S. Pat. No. 4,761,283; U.S. Pat. No. 5,360,897; U.S. Pat. No. 5,623,057; U.S. Pat. No. 5,847,112; US Patent Publication 2009/0017059; U.S. Patent Publication 200/0305127; U.S. Patent Publication 2009/0017072; and U.S. Patent Publication 2009/0010959).

The term “capsular polysaccharide from” S. pneumoniae is intended to encompass polysaccharide isolated from the native capsular polysaccharide (including modified forms thereof, e.g., to enhance immunogenicity, to facilitate conjugation and/or handling) as well as isolated or partially or completely synthetic multimers of a basic carbohydrate unit found in the native S. pneumoniae capsule (including modified forms thereof, e.g., to enhance immunogenicity, to facilitate conjugation and/or handling).

In embodiments of the invention, the capsular polysaccharide comprises fragments of the native capsular polymers (i.e., not the intact capsular polysaccharides); while not wishing to be bound by any theory of the invention, fragments of capsular polysaccharide may have enhanced immunogenicity as compared with the intact native capsular polysaccharide (see, e.g., U.S. Pat. No. 4,761,283 and U.S. Pat. No. 5,360,897). The fragments can be prepared from the native capsular polysaccharides or can be partially or completely synthetic. Methods of depolymerizing (i.e., reducing the chain length) of polysaccharides are known in the art. Suitable methods include the use of hydrogen peroxide and/or acid hydrolysis.

Further, the polysaccharide can optionally be O-acetylated (for example, with the same O-acetylation pattern as in the native capsular polysaccharide) or it may be partially or completely de-O-acetylated at one or more positions of the polysaccharide rings and/or it may be partially or completely hyper-O-acetylated relative to the native capsular polysaccharide.

Defined compositions of S. pneumoniae capsular polysaccharides having characterized structural and physical properties are known in the art (see, e.g., U.S. Pat. No. 5,847,112 and U.S. Pat. No. 5,623,057). For example, a preparation of S. pneumoniae capsular polysaccharide can be partially hydrolyzed to a predetermined endpoint to maintain the antigenic properties of the capsular polysaccharide and the polydispersity decreased. To illustrate, the capsular polysaccharide preparation can comprise on average less than about 1000 oligosaccharide repeat units per molecule, polydispersities between about 1 and 1.4, intrinsic viscosities between about 0.6 and 3 dL/g, and less than about 3% contamination of type-specific polysaccharide by group-specific C-polysaccharide (see, e.g., U.S. Pat. No. 5,847,112).

In representative embodiments, the capsular polysaccharide has an average size (e.g., weight-average molecular weight; M_{w}) above about 80 kDa, 100 KDa, 200 kDa, 300 kDa, 400 kDa, 500 kDa or 1000 kDa, for example an average size of 50-1600, 80-1400, 100-1000, 150-500 or 200-400 kDa (note that where average size is M_{W}, “kDa” units should be replaced with “×10³”) (see, e.g., U.S. Patent Publication 2009/0017072).

### III. FLAGELLINS

Flagellin proteins are known and described, for example, in U.S. Patent Nos. 6,585,980, 6130,082; 5,888,810; 5,618,533; 4,886,748 and U.S. Patent Publication No. US 2003/0044429 A1; and Donnelly et al., (2002) J. Biol. Chem. 43: 40456. Most gram-negative bacteria express flagella, which are surface structures that provide motility. The flagella are formed from a basal body, a filament, and a hook that connects the two. The filament is formed of a long polymer of a single protein, flagellin, with a small cap protein at the end. Polymerization of flagellin is mediated by conserved regions at the N- and C-termini, whereas the intervening hypervariable region of the flagellin protein is very diverse in sequence and length among species.

The flagellin can be derived from flagellins from any suitable source. A number of flagellin genes have been cloned and sequenced (see, e.g., Kuwajima et al., (1986) J. Bact. 168:1479; Wei et al., (1985) J. Mol. Biol. 186:791-803; and Gill et al., (1983) J. Biol. Chem. 258:7395-7401). Non-limiting sources of flagellins include but are not limited to S. enteritidis, S. typhimurium, S. dublin, H. pylori, V. cholera, S. marcesens, S. flexneri, S. enterica, T. pallidum, L. pneumophila, B. burgdorferei, C. difficile, A. tumefaciens, R. meliloti, B. clarridgeiae, R. lupine, P. mirabilis, B. subtilis, P. aeruginosa, and E. coli.

The N-terminal and C-terminal constant regions of flagellin are well characterized in the art and have been described, for example, in Mimori-Kiyosue et al., (1997) J. Mol. Virol. 270:222-237; lino et al., (1977) Ann. Rev. Genet. 11:161-182; and Schoenhals et al, (1993) J. Bacteriol. 175:5395-5402. As is understood by those skilled in the art, the size of the constant regions will vary somewhat depending on the source of the flagellin protein. In general, the N-terminal constant domain includes the approximately 170 or 180 N-terminal amino acids of the protein, whereas the C-terminal constant domain typically spans the approximately 85 to 100 C-terminal amino acids. The central hypervariable region varies considerably by size and sequence among bacteria, and accounts for most of the difference in molecular mass. The N- and C-terminal constant regions of flagellin proteins from a variety of bacteria are known, and others can be readily identified by those skilled in the art using known alignment techniques, which are facilitated by the elucidation of the crystal structure of the flagellin monomer (Samatey et al., (2001) Nature 41:331).

The terms “flagellin,” “flagellin N-terminal constant region” and “flagellin C-terminal constant region” include active fragments and modifications of any of the foregoing, which include for example, modifications that enhance the immune response to the S. pneumoniae antigen (e.g., by activating the TLR5 pathway). As further illustrations, the native flagellin or flagellin regions can be modified to increase safety and/or immune response and/or as a result of cloning procedures or other laboratory manipulations. In some embodiments, the flagellin comprises the full-length flagellin or, alternatively, can comprise an active fragment thereof. Further, the terms “flagellin,” “flagellin N-terminal constant region” and “flagellin C-terminal constant region” and like terms include polypeptides that comprise, consist essentially of, or consist of the naturally occurring amino acid sequences and further encompass polypeptides that comprise, consist essentially of, or consist of an amino acid sequence that is substantially identical or similar to the amino acid sequence of a naturally occurring flagellin, flagellin N-terminal constant region or flagellin C-terminal constant region, respectively, or an active fragment thereof.

As used herein, an “active fragment” of a flagellin, flagellin N-terminal constant region, C-terminal constant region, or any other flagellin region is a fragment of at least about 50, 75, 100, 125, 150, 200, 250 or 300 or more contiguous amino acids and/or less than about 300, 250, 200, 150, 125, 100 or 75 contiguous amino acids, including any combination thereof as long as the lower limit is less than the upper limit, where the active fragment enhances the immune response (optionally, a protective immune response) to the S. pneumoniae antigen in a host (e.g., by activating the TLR5 pathway). In particular embodiments, the active fragment enhances the immune response (optionally a protective immune response) to the S. pneumoniae antigen at least about 50%, 75%, 80%, 85%, 90%, or 95% or more of the level observed with the full-length flagellin or flagellin region, or enhances the immune response to the same or essentially the same extent as the full-length flagellin or flagellin region or enhances the immune response to an even greater extent than the full-length flagellin or flagellin region. Methods of measuring the immune response are well-known in the art (e.g., measurement of antigen-specific IgG). Further, in embodiments of the invention an “active fragment” of a flagellin, flagellin N-terminal constant region, C-terminal constant region, or any other flagellin domain induces an immune response (optionally a protective immune response) in a host against S. pneumoniae (e.g., IgG that react with S. pneumoniae), that is at least about 50%, 75%, 80%, 85%, 90%, or 95% or more of the immune response induced by the full-length flagellin or flagellin region, or induces an immune response that is the same as or essentially the same as the full-length flagellin or flagellin region or induces an immune response that is even greater than the immune response induced by the full-length flagellin or flagellin region.

In embodiments of the invention, a “modified” flagellin, flagellin N-terminal constant region, C-terminal constant region, or any other flagellin region (and similar terms) enhances the immune response (optionally a protective immune response) to the S. pneumoniae antigen to at least about 50%, 75%, 80%, 85%, 90%, or 95% or more of the level of enhancement observed with the native flagellin or flagellin region, or enhances the immune response to the same or essentially the same extent as the native flagellin or flagellin region or enhances the immune response to an even greater extent than the native flagellin or flagellin region. Methods of measuring the immune response are well-known in the art (e.g., measurement of antigen-specific IgG). Further, in embodiments of the invention a “modified” flagellin, flagellin N-terminal constant region, C-terminal constant region, or any other flagellin region induces an immune response (optionally a protective immune response) in a host against S. pneumoniae (e.g., IgG that react with S. pneumoniae), that is at least about 50%, 75%, 80%, 85%, 90%, or 95% or more of the immune response induced by the native flagellin or flagellin region, or induces an immune response that is the same as or essentially the same as the native flagellin or flagellin region or induces an immune response that is even greater than the immune response induced by the native flagellin or flagellin region.

A great deal of structure/function characterization of flagellin proteins has been reported in the literature. Those skilled in the art will be able to identify other suitable flagellin adjuvants within the scope of the present invention, in addition to those specifically disclosed herein, using no more than routine skill. For example, the circulating IgG titers against an antigen following administration of a flagellin fusion protein or flagellin composition (i.e., flagellin+antigen) of the invention can be compared with the circulating IgG induced by administration of the antigen alone.

Generally, the flagellin N-terminal and/or C-terminal constant region comprises a TLR5 recognition site(s) and is able to activate the TLR5 pathway. Regions of the flagellin protein involved in TLR5 signaling have been identified by Smith et al. (2003) Nat. Immunol. 4:1247-1253 (e.g., amino acids 78-129, 135-173 and 394-444 of S. typhimurium flagellin or orthologs or modified forms thereof). Further, in representative embodiments, the N-terminal constant region comprises the N-terminal RINSA domain (amino acids 31-52 of the S. dublin flagellin) as described by Eaves-Pyles et al. (2001) J. Immunology 167: 7009-7016, or an ortholog or modified form thereof that enhances the immunogenicity of the S. pneumoniae antigen.

In other embodiments, the N-terminal constant region comprises the D1 and D2 domains, and the C-terminal constant region comprises the D1 and D2 domains (Eaves-Pyles et al. (2001) J. Immunology 167: 7009-7016) or a modified form thereof.

In still further representative embodiments, the flagellin N-terminal and/or C-terminal constant region comprises the peptide GAVQNRFNSAIT (SEQ ID NO:1) as described by U.S. Patent Publication No. US 2003/0044429 A1 to Alderem et al., or an ortholog or modification thereof.

In still other embodiments, the N-terminal constant domain comprises the “motif N” (e.g., amino acids 98-108 of the S. muenchen flagellin) and/or the C-terminal constant domain comprises the “motif C” (e.g., amino acids 441-449 of S. muenchen flagellin) identified by Kanneganti et al., (2004) J. Biol. Chem. 279:5667-5676, or an ortholog or modified form thereof that enhances an immune response to the S. pneumoniae antigen.

In other illustrative embodiments, the N-terminal constant domain comprises amino acids 88 to 97 of the P. aeruginosa flagellin (see, e.g., Verma et al., (2005) Infect. Immun. 73:8237-8246) or an ortholog or modified form thereof.

In some embodiments of the invention, the flagellin hypervariable region between the constant regions is deleted (in whole or in part); in other embodiments the hypervariable region is present.

Further, the flagellin adjuvant can comprise a hinge region between the N-terminal constant and C-terminal constant regions. The hypervariable region or a S. pneumoniae antigen(s) can function as a hinge region. Additionally, or alternatively, a segment of about 2, 3, 4, 6, 8, 10, 15, 20, 30, 50 or more amino acids can function as a hinge region.

Optionally, the flagellin adjuvant can be a fusion protein comprising any other polypeptide of interest. For example, the flagellin adjuvant can be a fusion protein comprising one or more S. pneumoniae antigens and/or one or more antigens from another organism(s) (e.g., bacterial, viral, protozoan, yeast or fungal) and/or any other polypeptide of interest. In embodiments of the invention, the fusion protein comprises a S. pneumoniae PspA1 antigen, a S. pneumoniae PspA2 antigen, a S. pneumoniae PspA3 antigen, a S. pneumoniae PspA4 antigen, a S. pneumoniae PspA5 antigen and/or a S. pneumoniae PspA6 antigen. In exemplary embodiments, the fusion protein comprises two or more (i.e., two, three, four, five or six) of a S. pneumoniae PspA1 antigen, a S. pneumoniae PspA2 antigen, a S. pneumoniae PspA3 antigen, a S. pneumoniae PspA4 antigen, a S. pneumoniae PspA5 antigen and/or a S. pneumoniae PspA6 antigen.

In representative embodiments, the flagellin adjuvant can be a fusion protein comprising an immunomodulatory compound. For example, it is known in the art that immune responses can be enhanced by an immunomodulatory cytokine or chemokine (e.g., α-interferon, β-interferon, γ-interferon, ω-interferon, τ-interferon, interleukin-1α, interleukin-1β, interleukin-2, interleukin-3, interleukin-4, interleukin 5, interleukin-6, interleukin-7, interleukin-8, interleukin-9, interleukin-10, interleukin-11, interleukin 12, interleukin-13, interleukin-14, interleukin-18, B cell growth factor, CD40 Ligand, tumor necrosis factor-α, tumor necrosis factor-β, monocyte chemoattractant protein-1, granulocyte-macrophage colony stimulating factor, lymphotoxin, CCL25 [MECK], and CCL28 [TECH]) or active fragments thereof.

### IV. FLAGELLIN FUSION PROTEINS

The invention further provides a fusion protein comprising a flagellin adjuvant and one or more S. pneumoniae polypeptide antigens. Flagellin adjuvants and S. pneumoniae polypeptide antigens are described herein.

In representative embodiments, the invention provides a fusion protein comprising a flagellin adjuvant and one or more of a S. pneumoniae PspA antigen, a S. pneumoniae PsaA antigen, a S. pneumoniae PspC antigen (optionally, a transmembrane deletion form), a S. pneumoniae PcpA antigen, a S. pneumoniae PdB antigen, a S. pneumoniae histidine triad protein antigen (PhtA, PhtB, PhtD and/or PhtE), a S. pneumoniae pilus protein subunit antigen, a S. pneumoniae pneumolysin antigen (optionally a detoxified form), a S. pneumoniae PiuA antigen, a S. pneumoniae PiaA antigen, a S. pneumoniae choline binding protein antigen (optionally, a transmembrane deletion form), a S. pneumoniae CbpA antigen (optionally, a transmembrane deletion form), a S. pneumoniae glyceraldehyde-3-phospate dehydrogenase antigen, a S. pneumoniae M like protein antigen, a S. pneumoniae HSP70 antigen, and a S. pneumoniae Usp45 antigen. In exemplary embodiments, the fusion protein comprises a S. pneumoniae PspA antigen and a S. pneumoniae PsaA antigen. In other representative embodiments, the fusion protein comprises a S. pneumoniae PiuA antigen and a S. pneumoniae PiaA antigen. In further illustrative embodiments, the fusion protein comprises a flagellin adjuvant and a S. pneumoniae PspA1 antigen, S. pneumoniae PspA2 antigen, a S. pneumoniae PspA3 antigen, a S. pneumoniae PspA4 antigen, a S. pneumoniae PspA5 antigen and/or a S. pneumoniae PspA6 antigen. In embodiments of the invention, the fusion protein comprises two or more of a S. pneumoniae PspA1 antigen, a S. pneumoniae PspA2 antigen, a S. pneumoniae PspA3 antigen, a S. pneumoniae PspA4 antigen, a S. pneumoniae PspA5 antigen and/or a S. pneumoniae PspA6 antigen, which two or more antigens may optionally form a fusion peptide.

The fusion protein can further comprise one or more additional flagellin adjuvants, S. pneumoniae antigens, and/or antigens from another organism (e.g., bacterial, viral, protozoan, yeast or fungal).

The S. pneumoniae antigen(s) can be fused to the flagellin adjuvant in any suitable configuration, with or without intervening sequence(s). For example, one or more S. pneumoniae antigen(s) can be an N-terminal extension of the flagellin adjuvant (optionally with an intervening sequence(s)); in embodiments, the S. pneumoniae antigen makes up the amino-terminal portion of the fusion protein. As another option, one or more S. pneumoniae antigen(s) can be a C-terminal extension of the flagellin adjuvant (optionally with an intervening sequence(s)); in embodiments, the S. pneumoniae antigen makes up the carboxy-terminal portion of the fusion protein. Further, one or more of the S. pneumoniae antigen(s) can be inserted into the flagellin adjuvant (e.g., between the N-terminal and C-terminal constant regions). If multiple S. pneumoniae antigens are present, they can be incorporated into different sites in the fusion protein. For example, one antigen can be an N-terminal extension and one can be inserted into the protein sequence of the flagellin adjuvant (e.g., between the N-terminal and C-terminal constant regions). As another nonlimiting example, one antigen can be a C-terminal extension and one can be inserted into the protein sequence of the flagellin adjuvant (e.g., between the N-terminal and C-terminal constant regions). As yet another option, one antigen can be an N-terminal extension and the other can be a C-terminal extension. In embodiments of the invention, a fusion peptide comprising two or more S. pneumoniae antigens is incorporated as an N-terminal extension, a C-terminal extension and/or can be inserted into the protein coding sequence of the flagellin adjuvant (e.g., between the N-terminal and C-terminal constant regions). Further, when there are multiple flagellin adjuvants, the antigen(s) need not be positioned in the same location in each of the flagellin adjuvants.

In embodiments wherein the S. pneumoniae antigen(s) is located between the N-terminal constant region and the C-terminal constant region of the flagellin adjuvant, the S. pneumoniae antigen(s) can further be located between the N-terminal constant region and the hypervariable region and/or between the hypervariable region and the C-terminal constant region and/or inserted into the hypervariable region. Further, the hypervariable region can be partially or completely deleted. When the antigen(s) is positioned between one of the constant regions and the hypervariable region or is inserted into the hypervariable region, the sequences need not be directly fused to each other, i.e., there may be an intervening sequence.

As described above, the flagellin adjuvant can comprise a hinge region between the N-terminal constant and C-terminal constant regions. The hypervariable region and/or the S. pneumoniae antigen(s) can function as a hinge region. Additionally, or alternatively, a segment of about 1, 2, 3, 4, 6, 8, 10, 15, 20, 30, 50 or more amino acids can function as a hinge region.

A non-limiting example of a fusion protein of the invention is provided in the working Examples herein. Additional fusion proteins beyond those specifically disclosed herein can be routinely identified by those skilled in the art.

Unless indicated otherwise, flagellin and flagellin fusion proteins of the invention are administered per se as a polypeptide (or a nucleic acid encoding the polypeptide) and not as part of a live, killed, or recombinant bacterium- or virus-vectored vaccine. Further, unless indicated otherwise, the flagellins and flagellin fusion proteins of the invention are isolated flagellins and flagellin fusion proteins, e.g., are not incorporated into flagella.

### V. RECOMBINANT NUCLEIC ACIDS AND PRODUCTION OF FUSION PROTEINS

As used herein, the term “nucleic acid” encompasses both RNA and DNA, including cDNA, genomic DNA, synthetic (e.g., chemically synthesized) DNA and chimeras of RNA and DNA. The nucleic acid may be double-stranded or single-stranded. The nucleic acid may be synthesized using nucleotide analogs or derivatives (e.g., inosine or phosphorothioate nucleotides). Such nucleotides can be used, for example, to prepare nucleic acids that have altered base-pairing abilities or increased resistance to nucleases.

The fusion proteins of the invention can be produced in, and optionally purified from, cultured cells or organisms expressing a heterologous nucleic acid encoding the fusion protein for a variety of purposes (e.g., to produce immunogenic formulations, as a diagnostic or research reagent, and the like).

In some embodiments, the fusion protein can be collected and, optionally, purified from the host cell. For example, the fusion protein can be collected from the conditioned medium. According to this embodiment, it may be advantageous to express the fusion protein operably associated with a secretory signal sequence. Alternatively, the fusion protein can be isolated from the host cell (e.g., the host cell can be lysed and the fusion protein isolated therefrom).

In other embodiments, the host cells are collected and the fusion protein is not isolated therefrom.

Unless indicated otherwise, the flagellins and fusion proteins of the invention are isolated and not expressed as part of flagella (i.e., are not incorporated into flagella).

Generally, the heterologous nucleic acid is incorporated into an expression vector (viral or non-viral). Suitable expression vectors include but are not limited to plasmids, bacteriophage, bacterial artificial chromosomes (bacs), yeast artificial chromosomes (yacs), cosmids, virus vectors, and the like. Expression vectors compatible with various host cells are well known in the art and contain suitable elements for transcription and translation of nucleic acids. Typically, an expression vector contains an “expression cassette,” which includes, in the 5′ to 3′ direction, a promoter, a coding sequence encoding the fusion protein operatively associated with the promoter, and, optionally, a termination sequence including a stop signal for RNA polymerase and a polyadenylation signal for polyadenylase.

Expression vectors can be designed for expression of polypeptides in prokaryotic or eukaryotic cells. For example, polypeptides can be expressed in bacterial cells such as E. coli, insect cells (e.g., in the baculovirus expression system), yeast cells, mammalian cells, or plant cells. Examples of vectors for expression in yeast S. cerevisiae include pYepSecl (Baldari et al., (1987) EMBO J. 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, Calif.). Baculovirus vectors available for expression of nucleic acids to produce proteins in cultured insect cells (e.g., Sf9 cells) include the pAc series (Smith et al., (1983) Mol. Cell. Biol. 3:2156-2165) and the pVL series (Lucklow, V. A., and Summers, M. d. (1989) Virology 170:31-39).

Additionally, the expression vector will generally include expression control sequences (e.g., transcription/translation control signals and polyadenylation signals), which are operably associated with the nucleic acid sequence encoding the fusion protein of the invention. It will be appreciated that a variety of promoter/enhancer elements can be used depending on the level and tissue-specific expression desired. The promoter can be constitutive or inducible (e.g., the metallothionein promoter or a hormone inducible promoter), depending on the pattern of expression desired. The promoter can be native or foreign and can be a natural or a partially or completely synthetic sequence. By foreign, it is intended that the promoter is not naturally occurring in the host cell into which the nucleic acid is introduced. The promoter is chosen so that it will function in the target cell(s) of interest. Moreover, specific initiation signals are generally provided for efficient translation of inserted protein coding sequences. These translational control sequences, which can include the ATG initiation codon and adjacent sequences, can be of a variety of origins, both natural and synthetic. In embodiments of the invention wherein the expression vector comprises two open reading frames to be transcribed, the open reading frames can be operatively associated with separate promoters or with a single upstream promoter and one or more downstream internal ribosome entry site (IRES) sequences (e.g., the picornavirus EMC IRES sequence).

Examples of mammalian expression vectors include pCDM8 (Seed, (1987) Nature 329:840) and pMT2PC (Kaufman et al. (1987), EMBO J. 6:187-195). When used in mammalian cells, the expression vector's control functions are often provided by viral regulatory elements. For example, commonly used promoters are derived from polyoma, adenovirus 2, cytomegalovirus and Simian Virus 40.

The invention further provides a host cell comprising (transiently or stably) a nucleic acid encoding a fusion protein of the invention. Suitable host cells are well-known in the art and include prokaryotic and eukaryotic cells. See, e.g., Goeddel, Gene Expression Technology: Methods in Enzymology 185, Academic Press, San Diego, Calif. (1990). It is well-known that proteins can be expressed in bacterial cells such as E. coli, insect cells (e.g., Sf9 cells), yeast cells, plant cells or mammalian cells (e.g., human, rat, mouse, hamster, bovine, porcine, ovine, caprine, equine, feline, canine, lagomorph, simian and the like). The host cell can be a cultured cell such as a cell of a primary or immortalized cell line. The host cell can be a cell in a microorganism, animal or plant being used essentially as a bioreactor. In particular embodiments of the present invention, the host cell is an, insect cell that allows for replication of expression vectors. For example, the host cell can be from Spodoptera frugiperda, such as the Sf9 or Sf21 cell lines, Drosophila cell lines, or mosquito cell lines, e.g., Aedes albopictus derived cell lines. Use of insect cells for expression of heterologous proteins is well documented, as are methods of introducing nucleic acids, such as vectors, e.g., insect-cell compatible vectors (such as baculovirus vectors), into such cells and methods of maintaining such cells in culture. See, for example, Methods in Molecular Biology, ed. Richard, Humana Press, NJ (1995); O'Reilly et al., Baculovirus Expression Vectors, A Laboratory Manual, Oxford Univ. Press (1994); Samulski et al., J. Virol. 63:3822-8 (1989); Kajigaya et al., Proc. Nat'l. Acad. Sci. USA 88: 4646-50 (1991); Ruffing et al., J. Virol. 66:6922-30 (1992); Kimbauer et al., Virology 219:37-44 (1996); Zhao et al., Virology 272:382-93 (2000); and U.S. Pat. No. 6,204,059 to Samulski et al. In particular embodiments of the present invention, the insect cell is an Sf9 cell.

Vectors can be introduced into prokaryotic or eukaryotic cells via conventional transformation or transfection techniques. As used herein, the terms “transformation” and “transfection” refer to a variety of art-recognized techniques for introducing foreign nucleic acids (e.g., DNA) into a host cell, including calcium phosphate or calcium chloride co-precipitation, DEAE-dextran-mediated transfection, lipofection, electroporation, microinjection, DNA-loaded liposomes, lipofectamine-DNA complexes, cell sonication, gene bombardment using high velocity microprojectiles, and viral-mediated transfection. Suitable methods for transforming or transfecting host cells can be found in Sambrook et al. (Molecular Cloning: A Laboratory Manual, 2nd Edition, Cold Spring Harbor Laboratory press (1989)), and other laboratory manuals.

In further embodiments of the present invention, the host cell can be stably transformed with the heterologous nucleic acid sequence encoding the fusion protein. “Stable transformation” as used herein generally refers to the integration of the heterologous nucleic acid sequences into the genome of the host cell in contrast to “transient transformation” wherein the heterologous nucleic acid sequence introduced into the host cell does not integrate into the genome of the host cell. The term “stable transformant” can further refer to stable maintenance of an episome (e.g., an Epstein-Barr Virus (EBV) derived episome) in the cell.

When producing stably transformed cells, often only a small fraction of cells (in particular, mammalian cells) integrate a foreign nucleic acid into their genome. In order to identify and select these integrants, a nucleic acid that encodes a selectable marker (e.g., resistance to antibiotics) can be introduced into the host cells along with the nucleic acid of interest. Preferred selectable markers include those that confer resistance to drugs, such as G418, hygromycin and methotrexate. Nucleic acids encoding a selectable marker can be introduced into a host cell on the same vector as that comprising the nucleic acid of interest or can be introduced on a separate vector. Cells stably transfected with the introduced nucleic acid can be identified by drug selection (e.g., cells that have incorporated the selectable marker gene will survive, while the other cells die).

The fusion protein can also be produced in a transgenic plant in which the isolated nucleic acid encoding the fusion protein is inserted into the nuclear or plastidic genome or is maintained as a stable episomal element. Plant transformation is known as the art. See, in general, Methods in Enzymology Vol. 153 (“Recombinant DNA Part D”) 1987, Wu and Grossman Eds., Academic Press and European Patent Application EP 0 693 554.

Foreign nucleic acids can be introduced into plant cells or protoplasts by several methods. For example, nucleic acid can be mechanically transferred by microinjection directly into plant cells by use of micropipettes. Foreign nucleic acid can also be transferred into a plant cell by using polyethylene glycol which forms a precipitation complex with the genetic material that is taken up by the cell (Paszkowski et al. (1984) EMBO J. 3:2712-22). Foreign nucleic acid can be introduced into a plant cell by electroporation (Fromm et al. (1985) Proc. Natl. Acad. Sci. USA 82:5824). In this technique, plant protoplasts are electroporated in the presence of plasmids or nucleic acids containing the relevant genetic construct. Electrical impulses of high field strength reversibly permeabilize biomembranes allowing the introduction of the plasmids. Electroporated plant protoplasts reform the cell wall, divide, and form a plant callus. Selection of the transformed plant cells comprising the foreign nucleic acid can be accomplished using phenotypic markers.

Cauliflower mosaic virus (CaMV) can be used as a vector for introducing foreign nucleic acids into plant cells (Hohn et al. (1982) “Molecular Biology of Plant Tumors,” Academic Press, New York, pp. 549-560; Howell, U.S. Pat. No. 4,407,956). CaMV viral DNA genome is inserted into a parent bacterial plasmid creating a recombinant DNA molecule which can be propagated in bacteria. The recombinant plasmid can be further modified by introduction of the desired DNA sequence. The modified viral portion of the recombinant plasmid is then excised from the parent bacterial plasmid, and used to inoculate the plant cells or plants.

High velocity ballistic penetration by small particles can be used to introduce foreign nucleic acid into plant cells. Nucleic acid is disposed within the matrix of small beads or particles, or on the surface (Klein et al. (1987) Nature 327:70-73).

A nucleic acid can be introduced into a plant cell by infection of a plant cell with Agrobacterium tumefaciens or Agrobacterium rhizogenes transformed with the nucleic acid. Under appropriate conditions, the transformed plant cells are grown to form shoots, roots, and develop further into plants. The nucleic acids can be introduced into plant cells, for example, by means of the Ti plasmid of Agrobacterium tumefaciens. The Ti plasmid is transmitted to plant cells upon infection by Agrobacterium tumefaciens, and is stably integrated into the plant genome (Horsch et al., (1987) Science 227:1229-1231; Fraley et al. (1983) Proc. Natl. Acad. Sci. USA 80:4803).

The fusion protein can further comprise other polypeptides, for example, purification signals (such as poly-His, a FLAG epitope, c-myc, hemagglutinin and the like), detectable signals (e.g., such as a reporter protein including without limitation alkaline phosphatase, green fluorescent protein, glutathione-S-transferase, β-glucuronidase, β-galactosidase, luciferase, etc.) or other polypeptides (e.g., cytokines or other antigens from S. pneumoniae or other organisms).

### VI. CONJUGATES

The conjugates of the invention comprise a flagellin adjuvant covalently linked to one or more S. pneumoniae antigens. The covalent linkage can be a direct covalent linkage and/or can comprise cross-linking between the flagellin adjuvant and the one or more S. pneumoniae antigens. In representative embodiments, the covalent linkage comprises a linker between the flagellin adjuvant and the S. pneumoniae antigen.

The S. pneumoniae antigen can be any suitable antigen including without limitation polypeptides (including glyco-polypeptides and lipo-polypeptides), polysaccharides (including glycol-polysaccharides and lipo-polysaccharides), and nucleic acids. Methods of conjugating the S. pneumoniae antigen to the flagellin adjuvant are known in the art.

In embodiments of the invention, the S. pneumoniae antigen is a capsular polysaccharide from one or more serotypes of S. pneumoniae (as described in more detail herein).

In embodiments of the invention, the flagellin adjuvant is a fusion protein comprising any other polypeptide of interest, e.g., an immunomodulatory polypeptide and/or one or more S. pneumoniae antigens (e.g., a PspA antigen) and/or one more antigens from another organism. To illustrate, in embodiments of the invention, the conjugate comprises a flagellin adjuvant covalently linked to a capsular polysaccharide from one or more serotypes of S. pneumoniae, wherein the flagellin adjuvant is a fusion protein comprising a S. pneumoniae PspA antigen.

Methods of purifying capsular polysaccharides from S. pneumoniae are known in the art (see, e.g., U.S. Pat. No. 4,761,283; U.S. Pat. No. 5,360,897; U.S. Pat. No. 5,623,057; U.S. Pat. No. 5,847,112; US Patent Publication 2009/0017059; U.S. Patent Publication 200/0305127; U.S. Patent Publication 2009/0017072; and U.S. Patent Publication 2009/0010959), and capsular polysaccharides from S. pneumoniae are available from commercial sources (e.g., American Type Culture Collection).

In embodiments of the invention, each conjugate comprises capsular polysaccharide from two or more serotypes of S. pneumoniae. In other representative embodiments, capsular polysaccharide from each serotype is individually conjugated to the flagellin adjuvant (i.e., each conjugate comprises capsular polysaccharide from one serotype of S. pneumoniae).

The conjugates can be prepared with any suitable ratio of the flagellin adjuvant to the capsular polysaccharide, which may be optimized to enhance the immunogenicity of the antigen and/or the adjuvant activity of the flagellin adjuvant. In representative embodiments, a ratio of capsular polysaccharide to flagellin adjuvant (w/w) of greater than about 1:1 is used. In other embodiments, a ratio of capsular polysaccharide to flagellin adjuvant of less than about 1:1 is used. In still other embodiments, a ratio of capsular polysaccharide to flagellin adjuvant of about 1:1 is used. In representative embodiments, conjugates with a polysaccharide to flagellin adjuvant ratio (w/w) of between about 1:2, 1:3, 1:5, 1:10 or 1:15 (excess polypeptide) and about 2:1, 3:1, 5:1, 10:1 or 15:1 (excess polysaccharide) are used.

Further, in embodiments of the invention, the capsular polysaccharide is covalently linked to the N-terminus and/or C-terminus of the flagellin adjuvant.

Methods of conjugating polysaccharides to a carrier protein are also standard in the art, and any suitable method can be employed to conjugate the capsular polysaccharide to the flagellin adjuvant (see, e.g., U.S. Pat. No. 4,761,283; U.S. Pat. No. 5,360,897; U.S. Pat. No. 5,623,057; U.S. Pat. No. 5,847,112; US Patent Publication 2009/0017059; U.S. Patent Publication 200/0305127; U.S. Patent Publication 2009/0017072; and U.S. Patent Publication 2009/0010959).

In embodiments of the invention, the S. pneumoniae capsular polysaccharide is conjugated to the carrier protein via a linker, for instance a bifunctional linker. The linker is optionally heterobifunctional or homobifunctional, having for example a reactive amino group and a reactive carboxylic acid group, 2 reactive amino groups or two reactive carboxylic acid groups. The linker has for example between 4 and 20, 4 and 12, 5 and 10 carbon atoms. A possible linker is ADH. Other linkers include B-propionamido (WO 00/10599), nitrophenyl-ethylamine (Geyer et al (1979) Med. Microbiol. Immunol. 165; 171-288), haloalkyl halides (U.S. Pat. No. 4,057,685), glycosidic linkages (U.S. Pat. No. 4,673,574, U.S. Pat. No. 4,808,700), hexane diamine and 6-aminocaproic acid (U.S. Pat. No. 4,459,286).

The polysaccharide conjugates can be prepared by any suitable coupling technique. For example, the conjugation method may rely on activation of the polysaccharide with 1-cyano-4-dimethylamino pyridinium tetrafluoroborate (CDAP) to form a cyanate ester. The activated saccharide may thus be coupled directly or via a spacer (linker) group to an amino group on the carrier protein. For example, the spacer can be cystamine or cysteamine to give a thiolated polysaccharide which can be coupled to the carrier via a thioether linkage obtained after reaction with a maleimide-activated carrier protein (for example using GMBS) or a haloacetylated carrier protein (for example, using iodoacetimide [e.g. ethyl iodoacetimide HCl] or N-succinimidyl bromoacetate or STAB, or SIA, or SBAP). In representative embodiments, the cyanate ester (optionally made by CDAP chemistry) is coupled with hexane diamine or ADH and the amino-derivatized saccharide is conjugated to the carrier protein using carbodiimide (e.g., EDAC or EDC) chemistry via a carboxyl group on the protein carrier. Such conjugates are described in WO 93/15760, WO 95/08348 and WO 96/29094.

Other suitable techniques use carbodiimides, hydrazides, active esters, norborane, p-nitrobenzoic acid, N-hydroxysuccinimide, S-NHS, EDC, TSTU (see, e.g., WO 98/42721). In particular embodiments, conjugation may involve a carbonyl linker which may be formed by reaction of a free hydroxyl group of the polysaccharide with CDI (Bethell et al J. Biol. Chem. 1979, 254; 2572-4, Hearn et al J. Chromatogr. 1981. 218; 509-18) followed by reaction of with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

The conjugates can also be prepared by direct reductive amination methods as described, for example, in U.S. Pat. No. 4,365,170 (Jennings) and U.S. Pat. No. 4,673,574 (Anderson). Other illustrative methods are described in EP-0-161-188, EP-208375 and EP-0-477508.

A further conjugation method involves the coupling of a cyanogen bromide (or CDAP) activated polysaccharide derivatized with adipic acid dihydrazide (ADH) to the protein carrier by carbodiimide condensation (Chu C. et al Infect. Immunity, 1983 245 256), for example using EDAC.

As another possible conjugation method, a hydroxyl group (e.g., an activated hydroxyl group, for example, a hydroxyl group activated to make a cyanate ester [e.g. with CDAP]) on a polysaccharide is linked to an amino or carboxylic group on a protein either directly or indirectly (through a linker). Where a linker is present, a hydroxyl group on a polysaccharide is optionally linked to an amino group on a linker, for example by using CDAP conjugation. A further amino group in the linker for example ADH) may be conjugated to a carboxylic acid group on a protein, for example by using carbodiimide chemistry, for example by using EDAC. In an embodiment, the pneumococcal capsular polysaccharide is conjugated to the linker first before the linker is conjugated to the carrier protein. Alternatively the linker may be conjugated to the carrier before conjugation to the polysaccharide.

A combination of techniques may also be used, with some polysaccharide-protein conjugates being prepared by CDAP, and some by reductive amination.

In general the following types of chemical groups on a protein carrier can be used for coupling/conjugation:

A) Carboxyl (for instance via aspartic acid or glutamic acid). In one embodiment this group is linked to amino groups on polysaccharides directly or to an amino group on a linker with carbodiimide chemistry e.g. with EDAC.

B) Amino group (for instance via lysine). In one embodiment this group is linked to carboxyl groups on polysaccharides directly or to a carboxyl group on a linker with carbodiimide chemistry e.g. with EDAC. In another embodiment this group is linked to hydroxyl groups activated with CDAP or CNBr on polysaccharides directly or to such groups on a linker; to polysaccharides or linkers having an aldehyde group; to polysaccharides or linkers having a succinimide ester group.

C) Sulphydryl (for instance via cysteine). In one embodiment this group is linked to a bromo or chloro acetylated polysaccharide or linker with maleimide chemistry. In one embodiment this group is activated/modified with bis diazobenzidine.

D) Hydroxyl group (for instance via tyrosine). In one embodiment this group is activated/modified with bis diazobenzidine.

E) Imidazolyl group (for instance via histidine). In one embodiment this group is activated/modified with bis diazobenzidine.

F) Guanidyl group (for instance via arginine).

G) Indolyl group (for instance via tryptophan).

On a polysaccharide, in general, the following groups can be used for coupling: OH, COOH or NH2. Further, aldehyde groups can be generated after different treatments known in the art such as: periodate, acid hydrolysis, hydrogen peroxide, etc.

### Direct Coupling Approaches:

Polysaccharide-OH+CNBr or CDAP - - - >cyanate ester+NH2-Prot - - - >conjugate
Polysaccharide-aldehyde+NH2-Prot - - - >Schiff base+NaCNBH3 - - - >conjugate
Polysaccharide-COOH+NH2-Prot+EDAC - - - >conjugate
Polysaccharide-NH2+COOH-Prot+EDAC - - - >conjugate

### Indirect Coupling Via Spacer (Linker) Approaches:

Polysaccharide-OH+CNBr or CDAP - - - >cyanate ester+NH2 - - - NH2 - - - >polysaccharide - - - NH2+COOH-Prot+EDAC - - - >conjugate
Polysaccharide-OH+CNBr or CDAP - - - >cyanate ester+NH2 - - - SH - - - >polysaccharide - - - SH+SH-Prot (native Protein with an exposed cysteine or obtained after modification of amino groups of the protein by SPDP for instance) - - - >polysaccharide-S—S-Prot
Polysaccharide-OH+CNBr or CDAP - - - >cyanate ester+NH2 - - - SH - - - >polysaccharide - - - SH+maleimide-Prot (modification of amino groups) - - - >conjugate
Polysaccharide-OH+CNBr or CDAP - - - >cyanate ester+NH2 - - - SH - - - >polysaccharide-SH+haloacetylated-Prot - - - >Conjugate
Polysaccharide-COOH+EDAC+NH2 - - - NH2 - - - >polysaccharide - - - NH2+EDAC+COOH-Prot - - - >conjugate
Polysaccharide-COOH+EDAC+NH2 - - - SH - - - >polysaccharide - - - SH+SH-Prot (native Protein with an exposed cysteine or obtained after modification of amino groups of the protein by SPDP for instance) - - - >polysaccharide-S—S-Prot
Polysaccharide-COOH+EDAC+NH2 - - - SH - - - >polysaccharide - - - SH+maleimide-Prot (modification of amino groups) - - - >conjugate
Polysaccharide-COOH+EDAC+NH2 - - - SH - - - >Polysaccharide-SH+haloacetylated-Prot - - - >Conjugate
Polysaccharide-Aldehyde+NH2 - - - NH2 - - - >polysaccharide - - - NH2+EDAC+COOH-Prot - - - >conjugate
   Note: instead of EDAC above, any suitable carbodiimide may be used.

In representative embodiments, the chemical groups on the flagellin adjuvant that may generally be used for coupling with a polysaccharide are amino groups (for instance on lysine residues), COOH groups (for instance on aspartic and glutamic acid residues) and SH groups (if accessible) (for instance on cysteine residues).

### VII. COMPOSITIONS

The invention also contemplates compositions comprising a flagellin fusion protein and/or conjugate of the invention (each as described in more detail herein). The compositions can comprise one or more flagellin fusion proteins and/or one or more conjugates of the invention. Further, the composition can comprise a flagellin adjuvant(s) that is not fused to a S. pneumoniae antigen, a S. pneumoniae polypeptide antigen(s) that is not fused to a flagellin adjuvant, unconjugated S. pneumoniae capsular polysaccharide, S. pneumoniae capsular polysaccharide that is conjugated to a carrier protein other than flagellin, and/or free carrier protein other than flagellin that is not conjugated to a S. pneumoniae antigen (e.g., polysaccharide). In embodiments of the invention, the composition can comprise an antigen from any other organism of interest (including other bacteria, fungi, yeast, viruses). As a nonlimiting illustration, in representative embodiments, the composition comprises a flagellin fusion protein and/or a conjugate of the invention comprising an antigen from an organism other than S. pneumoniae.

In representative embodiments wherein the composition comprises free carrier protein, the free carrier protein comprises no more than about 1%, 2%, 5%, 10%, 15%, 20%, 25%, 30% or 35% of the total amount of carrier protein in the composition. In representative embodiments wherein the composition comprises unconjugated capsular polysaccharide from one or more serotypes of S. pneumoniae, the unconjugated carrier protein comprises no more than about 1%, 2%, 5%, 10%, 15%, 20%, 25%, 30% or 35% of the total amount of capsular polysaccharide in the composition. In embodiments of the invention, the composition comprises essentially no (i.e., a negligible or insignificant amount) or no free carrier protein and/or unconjugated capsular polysaccharide and/or essentially no (i.e., a negligible or insignificant amount) or no free carrier is added to the composition.

In embodiments of the invention, two or more S. pneumoniae antigens are provided as part of a flagellin fusion protein. The S. pneumoniae antigens can each be individually fused to a flagellin adjuvant (e.g., the composition contains two or more flagellin fusion proteins each comprising a S. pneumoniae antigen), multiple (two or more) S. pneumoniae antigens can be fused to a single flagellin fusion protein, or a combination of approaches can be used wherein the composition comprises one or more flagellin fusion proteins each comprising one S. pneumoniae antigen and one or more fusion proteins each comprising two or more S. pneumoniae antigens.

In embodiments of the invention, capsular polysaccharide from two or more S. pneumoniae serotypes are conjugated to a flagellin adjuvant. The polysaccharide from the two or more S. pneumoniae serotypes can each be individually conjugated to a flagellin adjuvant (e.g., the composition contains two or more conjugates each comprising polysaccharide from one S. pneumoniae serotype), polysaccharide from multiple (two or more) S. pneumoniae serotypes can be conjugated to a single flagellin adjuvant, or a combination of approaches can be used wherein the composition comprises one or more conjugates each comprising polysaccharide from one S. pneumoniae serotype and one or more conjugates each comprising polysaccharide from two or more S. pneumoniae serotypes. Further, the composition can comprise a conjugate comprising polysaccharide from two or more serotypes of S. pneumoniae, a second conjugate comprising polysaccharide from a second set of two or more serotypes of S. pneumoniae (wherein the serotypes in the second set can partially overlap with the first set), and so on.

In representative embodiments, the invention provides a composition comprising: (a) a flagellin fusion protein of the invention; and (b) capsular polysaccharide from one or more serotypes of S. pneumoniae. The polysaccharide can comprise, consist essentially of or consist of unconjugated polysaccharide and/or polysaccharide conjugated to a carrier protein (e.g., an immunogenic carrier protein). Immunogenic compositions comprising unconjugated polysaccharide from S. pneumoniae are known in the art (see, e.g., U.S. Pat. No. 5,623,057; U.S. Pat. No. 5,847,112; and U.S. Pat. No. 6,224,880). For example, the composition can comprise, consist essentially of or consist of the capsular polysaccharides from the 23 serotypes found in PNEUMOVAX® 23 (serotypes 1, 2, 3, 4, 5, 6B, 7F, 8, 9N, 9V, 10A, 11A, 12F, 14, 15B, 17F, 18C, 19A, 19F, 20, 22F, 23F and 33F) (Merck & Co.).

The polysaccharide can comprise, consist essentially of or consist of polysaccharide conjugated to a carrier protein (e.g., an immunogenic carrier protein). Any suitable carrier protein can be used to conjugate the polysaccharide. In representative embodiments, the carrier protein is a flagellin adjuvant, a diphtheria toxin or toxoid (including the detoxified CRM₁₉₇ protein), a tetanus toxin or toxoid (including fragment C), Haemophilus influenzae protein D (e.g., non-typeable H. influenzae protein D), the outer membrane protein complex of Neisseria meningitides (e.g., N. meningitides b) or a purified subunit thereof such as MIEP (also known as PorB protein), a member of the polyhistidine triad family (e.g., PhtA, PhtB, PhtD or PhtE) or a fragment or fusion protein thereof such as a PhtD or PhtE fusion (see, e.g., WO 01/98334 and WO 03/54007), a heat shock protein, a pertussis protein, a cytokine, a lymphokine, a growth factor or hormone, an artificial protein comprising multiple human CD4+ T cell epitopes, a S. pneumoniae PspA protein, an iron uptake protein, toxin A or B of C. difficile and/or any other carrier protein now known or later discovered in the art (see, e.g., U.S. Patent Publication 2009/0017072). In particular embodiments, the carrier protein is a flagellin adjuvant. In representative embodiments, the composition comprises capsular polysaccharide-diphtheria CRM₁₉₇ conjugates comprising, consisting essentially of or consisting of the pneumococcal capsular polysaccharide conjugates found in PREVNAR® (4, 6B, 9V, 14, 18C, 19F, and 23F) or PREVNAR 13® (1, 3, 4, 5, 6A, 6B, 7F, 9V, 14, 18C, 19A, 19F and 23F) (Wyeth). In representative embodiments, the composition comprises capsular polysaccharide-H. influenzae protein D conjugates comprising, consisting essentially of or consisting of the pneumococcal capsular polysaccharide conjugates found in SYNFLORIX® (1, 4, 5, 6B, 7F, 9V, 14, 180, 19F, and 23F) (GlaxoSmithKline).

The invention also provides a composition comprising: (a) a flagellin conjugate of the invention, and (b) a S. pneumoniae polypeptide antigen. The polypeptide antigen can be fused or unfused to a flagellin adjuvant.

As a further aspect, the invention provides a composition comprising: (a) a flagellin fusion protein of the invention; and (b) a flagellin conjugate of the invention.

In representative compositions of the invention, the flagellin fusion protein can comprise one or more S. pneumoniae PspA antigens. To illustrate, the composition can comprise one or more flagellin fusion proteins comprising a S. pneumoniae PspA1 antigen, a S. pneumoniae PspA2 antigen, a S. pneumoniae PspA3 antigen, a S. pneumoniae PspA4 antigen, a S. pneumoniae PspA5 antigen and/or a S. pneumoniae PspA6 antigen. In representative embodiments, each PspA antigen is provided by a separate flagellin fusion protein.

### VIII. METHODS OF ADMINISTRATION AND SUBJECTS

The present invention can be practiced for prophylactic and/or therapeutic purposes, in accordance with known techniques.

The invention can be practiced to produce an immune response against S. pneumoniae in a subject, optionally a protective immune response. With respect to a protective immune response, the present invention can be practiced prophylactically to prevent infection by S. pneumoniae. In other embodiments, the methods of the invention are practiced to treat a subject infected by S. pneumoniae.

Immunogenic formulations for use in the inventive methods are described below. Boosting dosages can further be administered over a time course of days, weeks, months or years. In chronic infection, initial high doses followed by boosting doses may be advantageous.

The present invention can be practiced for both medical and veterinary purposes. Subjects to be treated by the methods of the invention can include both avian and mammalian subjects, mammalian subjects including but not limited to humans, non-human primates (e.g., monkeys, baboons, and chimpanzees), dogs, cats, goats, horses, pigs, cattle, sheep, and the like, and laboratory animals (e.g., rats, mice, rabbits, gerbils, hamsters, and the like).

Suitable subjects include both males and females and subjects of all ages including infant, juvenile, adolescent, adult and geriatric subjects. Subjects may be treated for any purpose, such as for eliciting a protective immune response; for eliciting the production of antibodies in that subject, which antibodies can be collected and used for other purposes such as research or diagnostic purposes or for administering to other subjects to produce passive immunity therein, etc.

In embodiments of the invention, the subject is a child less than about 5 years of age. In other representative embodiments, the subject is a child less than about 2 years of age (e.g., a toddler or an infant). For example, in embodiments of the invention, the subject is a child less than about 2 years of age and is administered a conjugated capsular polysaccharide as described herein.

In particular embodiments, the subject is a child or adult human subject and is considered at risk for S. pneumoniae infection. At risk populations are known in the art and include, without limitation: patients who have chronic cerebrospinal fluid leakage (e.g., resulting from congenital lesions, skull fractures, or neurosurgical procedures), chronic cardiovascular disease (e.g., congestive heart failure or cardiomyopathy), chronic pulmonary disease (e.g., chronic obstructive pulmonary disease or emphysema), chronic liver disease (e.g., cirrhosis), diabetes, alcoholism, asthma (e.g., when it occurs with chronic bronchitis, emphysema or long-term use of systemic corticosteroids), and/or functional or anatomic asplenia (e.g., sickle cell disease or splenectomy). Other high risk populations include immunosuppressed patients (e.g, as a result of congenital immunodeficiency, human immunodeficiency virus infection, leukemia, lymphoma, multiple myeloma, Hodgkin's disease, generalized malignancy, organ or bone marrow transplantation, therapy with alkylating agents, antimetabolites or immunosuppressive therapy [including systemic corticosteroids], chronic renal failure and/or nephrotic syndrome).

In some embodiments the subjects are aged subjects, e.g., human subjects about 60, 65 or 70 years of age or older, where other adjuvants such as alum are generally less effective. In addition, aged subjects are generally considered at increased risk for S. pneumoniae infection.

Accordingly, in particular embodiments, the invention provides a method of producing an immune response against S. pneumoniae in a subject (e.g., mammalian subject), the method comprising administering a fusion protein, conjugate, composition and/or immunogenic formulation of the invention to the subject in an amount effective to produce an immune response in the subject against S. pneumoniae.

The invention further provides a method of protecting a subject (e.g., a mammalian subject) from infection with Streptococcus pneumoniae, the method comprising administering a fusion protein, conjugate, composition and/or immunogenic formulation of the invention to the subject in an amount effective to protect the subject from infection with S. pneumoniae. In representative embodiments, the method is practiced to prevent S. pneumoniae infection in the subject. In representative embodiments, the method is practiced to treat an existing S. pneumoniae infection in the subject. In particular embodiments, the invention is practiced to protect a subject or population of subjects (e.g., a child less than about five or less than about two years of age or aged subjects) from invasive pneumococcal disease (e.g., bacteremia and/or meningitis), S. pneumoniae induced pneumonia, and/or S. pneumoniae induced otitis media.

The invention also encompasses a method of enhancing a protective immune response to S. pneumoniae in a subject (e.g., mammalian subject), the method comprising administering a fusion protein, conjugate, composition and/or immunogenic formulation of the invention to the subject in an amount effective to enhance the protective immune response to S. pneumoniae in the subject.

The invention further contemplates a method of protecting a child (e.g., less than about five or less than about two years of age) from infection with S. pneumoniae, the method comprising administering a fusion protein, conjugate, composition or immunogenic formulation of the invention to the child in an amount effective to protect the child from infection with S. pneumoniae. According to representative embodiments, the method comprises administering a flagellin-polysaccharide conjugate of the invention and/or a composition or immunogenic formulation of the invention comprising a conjugated polysaccharide (e.g., a flagellin conjugate). In particular embodiments, the invention is practiced to protect the child or a population of children from invasive pneumococcal disease (e.g., bacteremia and/or meningitis), S. pneumoniae induced pneumonia, and/or S. pneumoniae induced otitis media.

Those skilled in the art will appreciate that the one or more booster dosages can be administered.

Further, the invention can be practiced to administer the priming and/or booster dosage(s).

For infant and toddler subjects, an exemplary dosage scheme is administration at about 2 months, about 4 months, about 6 months, and about 12 to 15 months. The fusion proteins, conjugates, compositions and/or immunogenic formulations of the invention can be administered at one or more of these time points. In the case of polysaccharide antigens, those skilled in the art will appreciate that conjugated capsular polysaccharides (e.g., a flagellin conjugate) are generally administered to this patient population because the immune systems of children less than about two years of age are unable to mount an effective response to unconjugated polysaccharide.

The methods of the invention can also comprise administering other immunogenic agents directed against S. pneumoniae or any other organism (e.g., bacterial, fungal, yeast, viral, protozoan) of interest. The additional immunogenic agents can be administered in the same composition as the flagellin fusion protein and/or flagellin conjugate of the invention. Alternatively, they can be administered in a separate composition, concurrently or serially. As used herein, the term “concurrent” or “concurrently” means sufficiently close in time to produce a combined effect (that is, simultaneously or two or more events occurring within a short time period before or after each other).

For example, the flagellin fusion proteins and/or flagellin conjugates of the invention can be used in conjunction with other immunogenic agents directed against S. pneumoniae. Such other immunogenic agents directed against S. pneumoniae can be administered in the same composition as the flagellin fusion protein and/or flagellin conjugate of the invention. Alternatively, they can be administered in a separate composition, concurrently or serially. As another option, where multiple vaccinations over time are used to provide protection, the flagellin fusion proteins, conjugates, compositions and/or immunogenic formulations of the invention can be administered at certain time points and other immunogenic agents (e.g., PREVNAR®, PREVNAR 13®, PNEUMOVAX® 23 or SYNFLORIX®) can be provided at other time points.

Administration can be by any route known in the art. As non-limiting examples, the route of administration can be by inhalation (e.g., oral and/or nasal inhalation), oral, buccal (e.g., sublingual), rectal, vaginal, topical (including administration to the airways), intraocular, transdermal, by parenteral (e.g., intramuscular [e.g., administration to skeletal muscle], intravenous, intra-arterial, intraperitoneal and the like), subcutaneous, intradermal, intrapleural, intracerebral, and/or intrathecal routes.

In particular embodiments, administration is to a mucosal surface, e.g., by intranasal, inhalation, intra-tracheal, oral, buccal (e.g., sublingual), intra-ocular, rectal or vaginal administration, and the like. In general, mucosal administration refers to delivery to a mucosal surface such as a surface of the respiratory tract, gastrointestinal tract, urinary tract, reproductive tract, etc.

Methods of administration to the respiratory tract include but are not limited to transmucosal, intranasal, inhalation, bronchoscopic administration, or intratracheal administration or administration to the lungs.

The fusion proteins, conjugates, compositions, and immunogenic formulations of the invention can be administered to the lungs of a subject by any suitable means, optionally by administering an aerosol suspension of respirable particles comprising the fusion protein, composition, or immunogenic formulation which the subject inhales. The respirable particles can be liquid or solid. Aerosols of liquid particles comprising a fusion protein, composition, or immunogenic formulation of the invention may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the can likewise be produced with any solid particulate medicament aerosol generator, by techniques known in the pharmaceutical art.

The fusion proteins of the invention can be delivered per se or by delivering a nucleic acid that encodes the fusion protein and is expressed in the subject to produce the fusion protein, such as described in U.S. Pat. No. 5,589,466 to Feigner et al.

Immunomodulatory compounds, such as immunomodulatory chemokines and cytokines (preferably, CTL inductive cytokines) can be administered concurrently to a subject.

Cytokines may be administered by any method known in the art. Exogenous cytokines may be administered to the subject, or alternatively, a nucleic acid encoding a cytokine may be delivered to the subject using a suitable vector, and the cytokine produced in vivo. In particular embodiments, the cytokine is provided as a part of a fusion protein of the invention. For example, a fusion protein comprising a flagellin adjuvant, a S. pneumoniae antigen, and an immunomodulatory cytokine (e.g., interferon-γ) can be administered.

In addition to their use for prophylactic or therapeutic purposes, the fusion proteins, conjugates, compositions, and immunogenic formulations of the present invention can be administered to subjects for the purpose of producing antibodies to a S. pneumoniae antigen (e.g., a PspA antigen or a capsular polysaccharide), which antibodies are in turn useful for research, diagnostic or therapeutic/prophylactic purposes (e.g., to provide passive immunity) in human and animal subjects.

### VI. PHARMACEUTICAL FORMULATIONS

The invention further provides pharmaceutical formulations (e.g., immunogenic formulations) comprising a fusion protein, conjugate or composition of the invention in a pharmaceutically acceptable carrier. In particular embodiments, the pharmaceutical composition is formulated for mucosal, intramuscular or subcutaneous delivery. By “pharmaceutically acceptable” it is meant a material that is not toxic or otherwise undesirable.

In representative embodiments, the fusion protein and/or conjugate is present in the pharmaceutical formulation in an “immunogenically effective” amount. An “immunogenically effective amount” is an amount that is sufficient to evoke an active immune response (i.e., cellular and/or humoral) in the subject to which the pharmaceutical formulation is administered, optionally a protective immune response (e.g., a prophylactic and/or therapeutic after onset of infection). The degree of protection conferred need not be complete or permanent, as long as the benefits of administering the pharmaceutical formulation outweigh any disadvantages thereof. Immunogenically effective amounts depend on the fusion protein and/or the conjugate, the manner of administration, the severity of the disease being treated, the general state of health of the subject, and the judgment of the prescribing physician and can be routinely determined by those skilled in the art.

As described above, unless indicated otherwise, the flagellins or flagellin fusion proteins of the invention are administered per se as a polypeptide (or a nucleic acid encoding the protein) and not as part of live, killed, or recombinant bacterium- or virus-vectored vaccine. Further, unless indicated otherwise, the flagellins and flagellin fusion proteins of the invention are isolated flagellins and flagellin fusion proteins, e.g., are not incorporated into flagella.

Likewise, unless indicated otherwise, the conjugates of the invention are administered per se and not as part of live, killed, or recombinant bacterium- or virus-vectored vaccine. Further, unless indicated otherwise, the conjugates of the invention are isolated conjugates, e.g., are not incorporated into flagella.

Dosages of pharmaceutically active compounds can be determined by methods known in the art, see, e.g., Remington's Pharmaceutical Sciences (Maack Publishing Co., Easton, Pa.). In particular embodiments, the dosage of the fusion proteins and/or conjugates of the present invention ranges from at least about 0.1, 0.5, 1, 5, 10, 15, 20, 25, 30, 50, 75, 100, 150, 200 or 250 μg to about 5, 10, 15, 20, 25, 30, 50, 75, 100, 150, 200, 250, 300, 500 or 1000 μg for a typical (e.g., 70 kg) subject (including any combination of the lower and upper dosages as long as the lower value is less than the upper value). The initial dose can be followed by one or more boosting dosages over weeks, months or years. In embodiments of the invention, S. pneumoniae capsular polysaccharide from each serotype is present in the pharmaceutical formulation at a dose from about 0.1, 0.5, 1, 5, 10, 15, 20, 25, 30, 50, 75, 100, 150, 200 or 250 μg to about 5, 10, 15, 20, 25, 30, 50, 75, 100, 150, 200, 250, 300, 500 or 1000 μg for a typical (e.g., 70 kg) subject (including any combination of the lower and upper dosages as long as the lower value is less than the upper value).

In embodiments of the invention, the fusion protein or conjugate is at least about 2-fold, 3-fold, 5-fold, 10-fold, 15-fold, 20-fold, 25-fold or 30-fold more active (e.g., in inducing antigen specific IgG) as the antigen (e.g., polypeptide or polysaccharide) alone. Accordingly, in embodiments, the dosage of the antigen provided as part of a fusion protein and/or conjugate of the invention is about 50% or less, 25% or less, 10% or less, 5% or less, 4% or less, 3% or less, 2% or less, or 1% or less of the dosage of the antigen alone to achieve the same immunogenic response against the antigen. Further, the quality of the antibodies produced by the fusion proteins and/or conjugates of the invention may be greater than those produced by the separate polypeptides, for example, in terms of IgG isotype and/or affinity of the antibodies for antigen. Moreover, the fusion proteins and/or conjugates may result in enhanced T cell dependent antibody response, T cell independent antibody response, levels of memory B cells and/or antibody dependent complement mediated cytotoxicity.

Optionally, the fusion protein and/or conjugate is present in an immunogenically effective amount, as defined herein. Further, in some embodiments, the flagellin adjuvant is present in an “adjuvant effective amount.” An “adjuvant effective amount” is an amount of the flagellin adjuvant that is sufficient to enhance or stimulate the active immune response (cellular and/or humoral, e.g., including antibody-dependent complement mediated cytotoxicity) mounted by the host against the S. pneumoniae antigen(s), optionally an active mucosal immune response. In particular embodiments, the active immune response (e.g., humoral and/or cellular immune response, e.g., including antibody-dependent complement mediated cytotoxicity) by the host is enhanced by at least about 2, 3, 4, 5, 10, 15, 20, 30, 40, 50, 60, 75, 100, 150, 500, 1000-fold or more. In other embodiments, an “adjuvant effective amount” is an amount of the flagellin adjuvant that reduces the amount of antigen required to achieve a specified level of immunity (cellular and/or humoral), optionally mucosal immunity, for example, a reduction of at least about 15%, 25%, 35%, 50%, 65%, 75%, 80%, 85%, 90%, 95%, 98% or more in the amount of antigen. As a further option, an “adjuvant effective amount” can refer to an amount of the flagellin adjuvant that accelerates the induction of the immune response in the host and/or reduces the need for booster immunizations to achieve protection. As yet another alternative, an “adjuvant effective amount” can be an amount that prolongs the time period over which an immune response, optionally a protective immune response, is sustained (e.g., by at least about a 2-fold, 3-fold, 5-fold, 10-fold, 20-fold longer time period or more).

Optionally, the pharmaceutical formulation can comprise one or more additional S. pneumoniae antigens, which may or not be present as a fusion protein and/or flagellin conjugate comprising the antigen(s). Further, the pharmaceutical formulation can comprise one or more antigens from another organism (e.g., bacterial, viral, protozoan, yeast or fungal), which may or may not be present as a fusion protein and/or flagellin conjugate.

The pharmaceutical formulations of the invention can optionally comprise other medicinal agents, pharmaceutical agents, stabilizing agents, buffers, carriers, diluents, salts, tonicity adjusting agents, wetting agents, and the like, for example, sodium acetate, sodium lactate, sodium chloride, potassium chloride, calcium chloride, sorbitan monolaurate, triethanolamine oleate, etc.

For injection, the carrier will typically be a liquid. For other methods of administration, the carrier may be either solid or liquid. For inhalation administration, the carrier will be respirable, and is typically in a solid or liquid particulate form.

While adjuvants beyond flagellin are generally not required, the composition can optionally comprise an additional adjuvant, such as complete or incomplete Freund's adjuvant, aluminum phosphate, aluminum hydroxide, alum, cytokines, TLR ligands, and the like. In embodiments of the invention, the adjuvant in the composition consists essentially of or consists of the flagellin adjuvant. In representative embodiments, the composition does not comprise an adjuvant other than the flagellin adjuvant.

The concentration of the fusion protein and/or conjugate in the pharmaceutical formulations can vary widely, e.g., from less than about 0.01% or 0.1% up to at least about 2% to as much as 20% to 50% or more by weight, and will be selected primarily by fluid volumes, viscosities, etc., in accordance with the particular mode of administration selected.

The fusion protein and/or conjugate can be formulated for administration in a pharmaceutical carrier in accordance with known techniques. See, e.g., Remington, The Science And Practice of Pharmacy (9^{th} Ed. 1995). In the manufacture of a pharmaceutical composition according to the invention, the polypeptide(s) (including physiologically acceptable salts thereof) is typically admixed with, inter alia, an acceptable carrier. The carrier can be a solid or a liquid, or both, and is optionally formulated with the compound as a unit-dose formulation, for example, a tablet. A variety of pharmaceutically acceptable aqueous carriers can be used, e.g., water, buffered water, 0.9% saline, 0.3% glycine, hyaluronic acid, pyrogen-free water, pyrogen-free phosphate-buffered saline solution, bacteriostatic water, or Cremophor EL[R] (BASF, Parsippany, N.J.), and the like. These compositions can be sterilized by conventional techniques. One or more fusion proteins can be incorporated in the formulations of the invention, which can be prepared by any of the well-known techniques of pharmacy.

The pharmaceutical formulations can be packaged for use as is, or lyophilized, the lyophilized preparation generally being combined with a sterile aqueous solution prior to administration. The compositions can further be packaged in unit/dose or multi-dose containers, for example, in sealed ampoules and vials.

The pharmaceutical formulations can be formulated for administration by any method known in the art according to conventional techniques of pharmacy. For example, the compositions can be formulated to be administered intranasally, by inhalation (e.g., oral inhalation), orally, buccally (e.g., sublingually), rectally, vaginally, topically, intrathecally, intraocularly, transdermally, by parenteral administration (e.g., intramuscular [e.g., skeletal muscle], intravenous, subcutaneous, intradermal, intrapleural, intracerebral and intra-arterial, intrathecal), or topically (e.g., to both skin and mucosal surfaces, including airway surfaces).

In particular embodiments, the pharmaceutical formulation is administered to a mucosal surface, e.g., by intranasal, inhalation, intratracheal, oral, buccal, rectal, vaginal or intra-ocular administration, and the like.

For intranasal or inhalation administration, the pharmaceutical formulation can be formulated as an aerosol (this term including both liquid and dry powder aerosols). For example, the pharmaceutical formulation can be provided in a finely divided form along with a surfactant and propellant. Typical percentages of the composition are 0.01-20% by weight, preferably 1-10%. The surfactant is generally nontoxic and soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1-20% by weight of the composition, preferably 0.25-5%. The balance of the composition is ordinarily propellant. A carrier can also be included, if desired, as with lecithin for intranasal delivery. Aerosols of liquid particles can be produced by any suitable means, such as with a pressure-driven aerosol nebulizer or an ultrasonic nebulizer, as is known to those of skill in the art. See, e.g., U.S. Pat. No. 4,501,729. Aerosols of solid particles can likewise be produced with any solid particulate medicament aerosol generator, by techniques known in the pharmaceutical art. Intranasal administration can also be by droplet administration to a nasal surface.

Injectable formulations can be prepared in conventional forms, either as liquid solutions or suspensions, solid forms suitable for solution or suspension in liquid prior to injection, or as emulsions. Alternatively, one can administer the pharmaceutical formulations in a local rather than systemic manner, for example, in a depot or sustained-release formulation.

Extemporaneous injection solutions and suspensions can be prepared from sterile powders, granules and tablets of the kind previously described. For example, an injectable, stable, sterile formulation of the invention in a unit dosage form in a sealed container can be provided. The formulation can be provided in the form of a lyophilizate, which can be reconstituted with a suitable pharmaceutically acceptable carrier to form a liquid composition suitable for injection into a subject. The unit dosage form can be from about 1 μg to about 10 grams of the formulation. When the formulation is substantially water-insoluble, a sufficient amount of emulsifying agent, which is pharmaceutically acceptable, can be included in sufficient quantity to emulsify the formulation in an aqueous carrier. One such useful emulsifying agent is phosphatidyl choline.

Pharmaceutical formulations suitable for oral administration can be presented in discrete units, such as capsules, cachets, lozenges, or tables, as a powder or granules; as a solution or a suspension in an aqueous or non-aqueous liquid; or as an oil-in-water or water-in-oil emulsion. Oral delivery can be performed by complexing a compound(s) of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the protein(s) and a suitable carrier (which may contain one or more accessory ingredients as noted above). In general, the pharmaceutical formulations are prepared by uniformly and intimately admixing the compound(s) with a liquid or finely divided solid carrier, or both, and then, if necessary, shaping the resulting mixture. For example, a tablet can be prepared by compressing or molding a powder or granules containing the protein(s), optionally with one or more accessory ingredients. Compressed tablets are prepared by compressing, in a suitable machine, the formulation in a free-flowing form, such as a powder or granules optionally mixed with a binder, lubricant, inert diluent, and/or surface active/dispersing agent(s). Molded tablets are made by molding, in a suitable machine, the powdered protein moistened with an inert liquid binder.

Pharmaceutical formulations suitable for buccal (sub-lingual) administration include lozenges comprising the compound(s) in a flavored base, usually sucrose and acacia or tragacanth; and pastilles comprising the compound(s) in an inert base such as gelatin and glycerin or sucrose and acacia.

Pharmaceutical formulations suitable for parenteral administration can comprise sterile aqueous and non-aqueous injection solutions of the proteins, which preparations are preferably isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical formulations suitable for rectal administration are preferably presented as unit dose suppositories. These can be prepared by admixing the protein(s) with one or more conventional solid carriers, such as for example, cocoa butter and then shaping the resulting mixture.

Pharmaceutical formulations suitable for topical application to the skin preferably take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical formulation of the present invention with a lipophilic reagent (e.g., DMSO) that is capable of passing into the skin.

Pharmaceutical formulations suitable for transdermal administration can be in the form of discrete patches adapted to remain in intimate contact with the epidermis of the subject for a prolonged period of time. Formulations suitable for transdermal administration can also be delivered by iontophoresis (see, for example, Pharmaceutical Research 3:318 (1986)) and typically take the form of an optionally buffered aqueous solution of the compound(s). Suitable formulations can comprise citrate or bis\tris buffer (pH 6) or ethanol/water and can contain from 0.1 to 0.2M active ingredient.

Further, the fusion protein and/or conjugate can be formulated as a liposomal formulation. The lipid layer employed can be of any conventional composition and can either contain cholesterol or can be cholesterol-free. The liposomes that are produced can be reduced in size, for example, through the use of standard sonication and homogenization techniques.

The liposomal formulations can be lyophilized to produce a lyophilizate which can be reconstituted with a pharmaceutically acceptable carrier, such as water, to regenerate a liposomal suspension.

The immunogenic formulations of the invention can optionally be sterile, and can further be provided in a closed pathogen-impermeable container.

The present invention is more particularly described in the following examples that are intended as illustrative only since numerous modifications and variations therein will be apparent to those skilled in the art.

### Example 1

### Generation and Immunization with PspA-Flagellin Fusion Proteins

DNA encoding S. pneumoniae PspA1 was prepared from genomic DNA of a pneumococcal strain expressing this form of PspA. The DNA encoding Salmonella enteriditis flagellin was previously cloned (Mizel et al., (2009) Clinical and Vaccine Immunology 16:21-28). Using a PCR strategy, the DNA encoding PspA was fused to the 5′ terminus of the flagellin DNA. The resultant fusion DNA encoding PspA-flagellin was ligated into the pET29a expression vector. The nucleotide (SEQ ID NO:2) and amino acid (SEC) ID NO:3) sequences of the PspA-flagellin are shown in Table 2 and Table 3, respectively. The protein was generated, purified and tested for toll-like receptor 5 (TLR5)-specific signaling. The fusion protein retained full signaling activity.

Groups of 7 female BALB/c mice (7 weeks of age) were immunized intramuscularly on days 0 and 28 with 1.05 micrograms of PspA, 3.5 micrograms of PspA, 3 micrograms PspA-flagellin (FIiC) fusion protein, or 10 micrograms of PspA-flagellin fusion protein. The doses of PspA are equivalent to the amount of PspA in 3 micrograms and 10 micrograms of the PspA-flagellin fusion proteins.

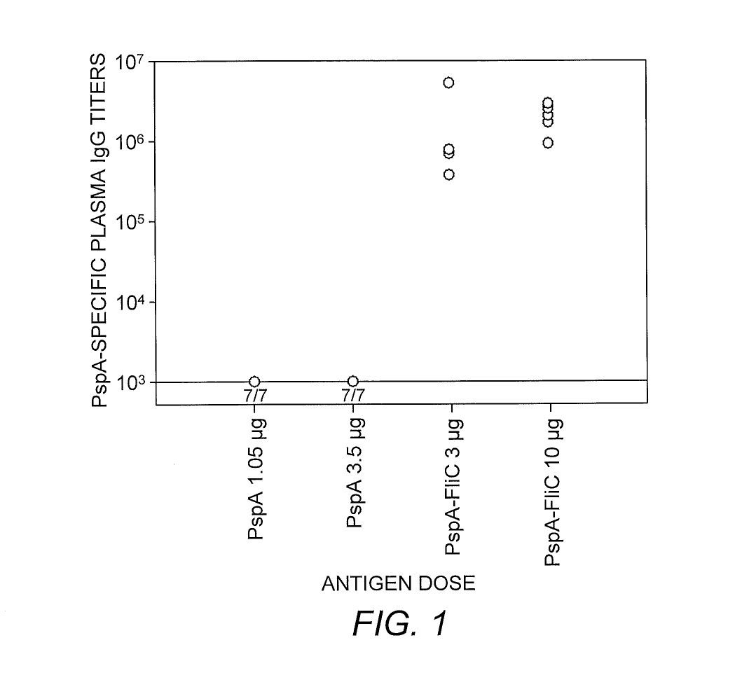

Ten days after the boost, the mice were bled and the plasma titers of anti-PspA IgG were determined by ELISA. The results are shown in FIG. 1. It is clear that the PspA by itself did not induce significant IgG production. However, when present in a fusion protein with flagellin, very high titers were achieved.

These studies are repeated with S. pneumoniae PspA2, PspA3, PspA4, PspA5 and/or PspA6 antigens. This study is also repeated by concurrently administering two or more of S. pneumoniae PspA1, PspA2, PspA3, PspA4, PspA5 and PspA6 antigens.

TABLE 2
PspA-Flagellin Chimera DNA Sequence (SEQ ID NO: 2)

ATGGAAGAATCTCCACAAGTTGTCGAAAAATCTTCATTAGAGAAGAAATATGAGGAAGCAAA
AGCAAAAGCTGATACTGCCAAGAAAGATTACGAAACGGCTAAAAAGAAAGCAGAAGACGCTC
AGAAAAAGTATGAAGATGATCAGAAGAGAACTGAGGAGAAAGCTCGAAAAGAAGCAGAAGCA
TCTCAAAAATTGAATGATGTGGCGCTTGTTGTTCAAAATGCATATAAAGAGTACCGAGAAGT
TCAAAATCAACGTAGTAAATATAAATCTGACGCTGAATATCAGAAAAAATTAACAGAGGTCG
ACTCTAAAATAGAGAAGGCTAGGAAAGAGCAACAGGACTTGCAAAATAAATTTAATGAAGTA
AGAGCAGTTGTAGTTCCTGAACCAAATGCGTTGGCTGAGACTAAGAAAAAAGCAGAAGAAGC
TAAAGCAGAAGAAAAAGTAGCTAAGAGAAAATATGATTATGCAACTCTAAAGGTAGCGCTAG
CGAAGAAAGAAGTAGAGGCTAAGGAACTTGAAATTGAAAAACTTCAATATGAAATTTCTACT
TTGGAACAAGAAGTTGCTACTGCTCAACATCAAGTAGATAATTTGAAAAAACTTCTTGCTGG
TGCGGATCCTGATGATGGCACAGAAGTTATAGAAGCTAAATTAAAAAAAGGAGAAGCTGAGC
TAAACGCTAAACAAGCTGAGTTAGCAAAAAAACAAACAGAACTTGAAAAACTTCTTGACGGC
CTTGATCCTGAAGGTAAGACTCAGGATGAATTAGATAAA [symbol] GCACAAGTCATTAATAC
AAACAGCCTGTCGCTGTTGACCCAGAATAACCTGAACAAATCTCAGTCCTCACTGAGTTCCG
CTATTGAGCGTCTGTCCTCTGGTCTGCGTATCAACAGCGCGAAAGACGATGCGGCAGGCCAG
GCGATTGCTAACCGCTTCACTTCTAATATCAAAGGTCTGACTCAGGCTTCCCGTAACGCTAA
CGACGGCATTTCTATTGCGCAGACCACTGAAGGTGCGCTGAATGAAATCAACAACAACCTGC
AGCGTGTGCGTGAGTTGTCTGTTCAGGCCACTAACGGGACTAACTCTGATTCCGATCTGAAA
TCTATCCAGGATGAAATTCAGCAACGTCTGGAAGAAATCGATCGCGTTTCTAATCAGACTCA
ATTTAACGGTGTTAAAGTCCTGTCTCAGGACAACCAGATGAAAATCCAGGTTGGTGCTAACG
ATGGTGAAACCATTACCATCGATCTGCAAAAAATTGATGTGAAAAGCCTTGGCCTTGATGGG
TTCAATGTTAATGGGCCAAAAGAAGCGACAGTGGGTGATCTGAAATCCAGCTTCAAGAATGT
TACGGGTTACGACACCTATGCAGCGGGTGCCGATAAATATCGTGTAGATATTAATTCCGGTG
CTGTAGTGACTGATGCAGCAGCACCGGATAAAGTATATGTAAATGCAGCAAACGGTCAGTTA
ACAACTGACGATGCGGAAAATAACACTGCGGTTGATCTCTTTAAGACCACTAAATCTACTGC
TGGTACCGCTGAAGCCAAAGCGATAGCTGGTGCCATTAAAGGTGGTAAGGAAGGAGATACCT
TTGATTATAAAGGCGTGACTTTTACTATTGATACAAAAACTGGTGATGACGGTAATGGTAAG
GTTTCTACTACCATCAATGGTGAAAAAGTTACGTTAACTGTCGCTGATATTGCCACTGGCGC
GACGGATGTTAATGCTGCTACCTTACAATCAAGCAAAAATGTTTATACATCTGTAGTGAACG
GTCAGTTTACTTTTGATGATAAAACCAAAAACGAGAGTGCGAAACTTTCTGATTTGGAAGCA
AACAATGCTGTTAAGGGCGAAAGTAAAATTACAGTAAATGGGGCTGAATATACTGCTAACGC
CACGGGTGATAAGATCACCTTAGCTGGCAAAACCATGTTTATTGATAAAACAGCTTCTGGCG
TAAGTACATTAATCAATGAAGACGCTGCCGCAGCCAAGAAAAGTACCGCTAACCCACTGGCT
TCAATTGATTCTGCATTGTCAAAAGTGGACGCAGTTCGTTCTTCTCTGGGGGCAATTCAAAA
CCGTTTTGATTCAGCCATTACCAACCTTGGCAATACGGTAACCAATCTGAACTCCGCGCGTA
GCCGTATCGAAGATGCTGACTATGCAACGGAAGTTTCTAATATGTCTAAAGCGCAGATTCTG
CAGCAGGCTGGTACTTCCGTTCTGGCGCAGGCTAACCAGGTTCCGCAAAACGTCCTCTCTTT
ACTGCGT [symbol] CACCACCACCACCACCACTGA
PspA = Underlined
Flagellin = Unaltered
Linker = Shaded
HIS tag = Italicized

TABLE 3
Amino Acid Sequence of PspA-Flagellin (SEQ ID NO: 3)

| | |
|---|---|
| MEESPQVVEKSSLEKKYEEAKAKADTAKKDYETAKKKAEDAQKKYEDDQK | 50 |
| RTEEKARKEAEASQKLNDVALVVQNAYKEYREVQNQRSKYKSDAEYQKKL | 100 |
| TEVDSKIEKARKEQQDLQNKFNEVRAVVVPEPNALAETKKKAEEAKAEEK | 150 |
| VAKRKYDYATLKVALAKKEVEAKELEIEKLQYEISTLEQEVATAQHQVDN | 200 |
| LKKLLAGADPDDGTEVIEAKLKKGEAELNAKQAELAKKQTELEKLLDGLD | 250 |
| PEGKTQDELDKKLAQVINTNSLSLLTQNNLNKSQSSLSSAIERLSSGLRI | 300 |
| NSAKDDAAGQAIANRFTSNIKGLTQASRNANDGISIAQTTEGALNEINNN | 350 |
| LQRVRELSVQATNGTNSDSDLKSIQDEIQQRLEEIDRVSNQTQFNGVKVL | 400 |
| SQDNQMKIQVGANDGETITIDLQKIDVKSLGLDGFNVNGPKEATVGDLKS | 450 |
| SFKNVTGYDTYAAGADKYRVDINSGAVVTDAAAPDKVYVNAANGQLTTDD | 500 |
| AENNTAVDLFKTTKSTAGTAEAKAIAGAIKGGKEGDTFDYKGVTFTIDTK | 550 |
| TGDDGNGKVSTTINGEKVTLTVADIATGATDVNAATLQSSKNVYTSVVNG | 600 |
| QFTFDDKTKNESAKLSDLEANNAVKGESKITVNGAEYTANATGDKITLAG | 650 |
| KTMFIDKTASGVSTLINEDAAAAKKSTANPLASIDSALSKVDAVRSSLGA | 700 |
| IQNRFDSAITNLGNTVTNLNSARSRIEDADYATEVSNMSKAQILQQAGTS | 750 |
| VLAQANQVPQNVLSLLRLEHHHHHH | |

### Example 2

### Generation and Immunization with Conjugated Capsular Polysaccharides from S. pneumoniae

Capsular polysaccharides are purified from one or more serotypes of S. pneumoniae or are purchased from commercial sources. Flagellin derived from S. enteritidis is expressed (see Example 1), and the capsular polysaccharides is individually chemically conjugated to the flagellin protein. The conjugate(s) is purified and tested for TLR5-specific signaling. Conjugates from different serotypes can optionally be mixed prior to testing in animals.

Groups of mice are immunized intramuscularly on days 0 and 28 with capsular polysaccharide alone or the flagellin conjugate(s). Ten days after the boost, the mice are bled and the plasma titers of IgG directed against the capsular polysaccharide are determined by ELISA. The T cell dependency of the humoral response is determined by comparison with mice that lack T cells. The generation of memory B cells is determined by assessing the numbers of plasma and memory cells.

### Example 3

### Immunization with PspA Fusion Proteins and Conjugated Capsular Polysaccharides

Four separate fusion proteins comprising S. pneumoniae PspA1, PspA2, PspA3 or PspA4 antigens are generated as illustrated in Example 1 for the PspA1 antigen such that all four fusion proteins are represented. Groups of mice are immunized intramuscularly on days 0 and 28 with the four fusion proteins and flagellin-capsular polysaccharide conjugate(s) as described in Example 2. The four fusion proteins and capsular polysaccharide are administered together in one composition or in separate compositions. The flagellin fusion protein(s) alone or flagellin-polysaccharide conjugates alone are administered to groups of mice for comparison purposes. Ten days after the boost, the mice are bled and the plasma titers of IgG directed against PspA1, PspA2, PspA3, PspA4 and capsular polysaccharide(s) are determined by ELISA. The T cell dependency of the humoral response is determined by comparison with mice that lack T cells. The generation of memory B cells can be determined by assessing the numbers of plasma and memory cells.

This experiment is repeated with the addition of a respiratory challenge with one or more serotypes of S. pneumoniae to evaluate the level of protection conferred by administration of the PspA antigens and/or polysaccharide conjugate(s). The challenge strain(s) is selected based on the PspA clade and origin of the polysaccharides used for immunization.

The foregoing is illustrative of the present invention, and is not to be construed as limiting thereof. The invention is defined by the following claims, with equivalents of the claims to be included therein.

1. A fusion protein comprising a flagellin adjuvant and a Streptococcus pneumoniae PspA antigen.

2. The fusion protein of claim 1, wherein the PspA antigen is a PspA1, PspA2, PspA3 and/or PspA4 antigen.

3. The fusion protein of claim 1, wherein the PspA antigen is a PspA1 antigen.

4. The fusion protein of claim 1, wherein the PspA antigen is a PspA2 antigen.

5. The fusion protein of claim 1, wherein the PspA antigen is a PspA3 antigen.

6. The fusion protein of claim 1, wherein the PspA antigen is a PspA4 antigen.

7. The fusion protein of claim 1, wherein the PspA antigen comprises two or more of a PspA1 antigen, a PspA2 antigen, a PspA3 antigen, and a PspA4 antigen.

8. The fusion protein of claim 1, wherein the PspA antigen is an amino terminal extension of the flagellin adjuvant.

9. The fusion protein of claim 1, wherein the PspA antigen is the amino-terminal portion of the fusion protein.

10. A nucleic acid encoding the fusion protein of claim 1.

11. A vector comprising the nucleic acid of claim 10.

12. A host cell comprising the nucleic acid of claim 10.

13. A method of making a fusion protein, the method comprising culturing the host cell of claim 12 in a culture medium under conditions sufficient for the fusion protein to be produced.

14. A conjugate comprising a flagellin adjuvant covalently linked to a capsular polysaccharide from one or more serotypes of Streptococcus pneumoniae.

15. The conjugate of claim 14, wherein the capsular polysaccharide is from 7 or more serotypes of S. pneumoniae.

16. The conjugate of claim 14, wherein the capsular polysaccharide is from 10 or more serotypes of S. pneumoniae.

17. The conjugate of claim 14, wherein the capsular polysaccharide is from 23 or more serotypes of S. pneumoniae.

18. The conjugate of claim 14, wherein the capsular polysaccharide comprises capsular polysaccharide from one or more of S. pneumoniae serotypes 4, 6B, 9V, 14, 18C, 19F and 23F.

19. The conjugate of claim 14, wherein the capsular polysaccharide comprises capsular polysaccharide from one or more of S. pneumoniae serotypes 1, 3, 4, 5, 6A, 6B, 7F, 9V, 14, 18C, 19A, 19F and 23F.

20. The conjugate of claim 14, wherein the capsular polysaccharide comprises capsular polysaccharide from one or more of S. pneumoniae serotypes 1, 4, 5, 6B, 7F, 9V, 14, 18C, 19F and 23F.

21. The conjugate of claim 14, wherein the capsular polysaccharide comprises capsular polysaccharide from one or more of S. pneumoniae serotypes 1, 2, 3, 4, 5, 6B, 7F, 8, 9N, 9V, 10A, 11A, 12F, 14, 15B, 17F, 18C, 19A, 19F, 20, 22F, 23F and 33F.

22. The conjugate of claim 14, wherein the capsular polysaccharide comprises capsular polysaccharide from one or more of S. pneumoniae serotypes 6B, 9V, 14, 19A, 19F and 23F.

23. A composition comprising:
(a) the fusion protein of claim 1; and
(b) a capsular polysaccharide from one or more serotypes of Streptococcus pneumoniae.

24. The composition of claim 23, wherein the capsular polysaccharide is not conjugated to a carrier protein.

25. The composition of claim 23, wherein the capsular polysaccharide is conjugated to a carrier protein.

26. The composition of claim 25, wherein the capsular polysaccharide is conjugated to a bacterial toxin or toxoid.

27. The composition of claim 25, wherein the capsular polysaccharide is conjugated to the diphtheria toxin CRM₁₉₇.

28. The composition of claim 25, wherein the capsular polysaccharide is conjugated to the outer membrane protein complex of Neisseria meningitides.

29. The composition of claim 25, wherein the capsular polysaccharide is conjugated to Haemophilus influenzae protein D.

30. A composition comprising:
(a) the fusion protein of claim 1; and
(b) a conjugate comprising a flagellin adjuvant covalently linked to a capsular polysaccharide from one or more serotypes of Streptococcus pneumoniae.

31. An immunogenic formulation comprising the fusion protein of claim 1.

32. A method of producing an immune response against Streptococcus pneumoniae in a mammalian subject, the method comprising administering the fusion protein of claim 1 to the mammalian subject in an amount effective to produce an immune response in the mammalian subject against Streptococcus pneumoniae.

33. A method of protecting a mammalian subject from infection with Streptococcus pneumoniae, the method comprising administering the fusion protein of claim 1 to the mammalian subject in an amount effective to protect the mammalian subject from infection with Streptococcus pneumoniae.

34. A method of enhancing a protective immune response to Streptococcus pneumoniae in a mammalian subject, the method comprising administering the fusion protein of claim 1 to the mammalian subject in an amount effective to enhance the protective immune response to Streptococcus pneumoniae in the mammalian subject

35-36. (canceled)

37. A method of protecting a child less than two years of age from infection with Streptococcus pneumoniae, the method comprising administering the composition of claim 25 to the child in an amount effective to protect the child from infection with Streptococcus pneumoniae.

38-39. (canceled)

40. An article of manufacture comprising a closed, pathogen-impermeable container and a sterile vaccine preparation enclosed within said container, wherein said vaccine preparation comprises the immunogenic formulation of claim 31.

41. A method of producing an immune response against Streptococcus pneumoniae in a mammalian subject, the method comprising administering the conjugate of claim 14 to the mammalian subject in an amount effective to produce an immune response in the mammalian subject against Streptococcus pneumoniae.

42. A method of protecting a mammalian subject from infection with Streptococcus pneumoniae, the method comprising administering the conjugate of claim 14 to the mammalian subject in an amount effective to protect the mammalian subject from infection with Streptococcus pneumoniae.

43. A method of enhancing a protective immune response to Streptococcus pneumoniae in a mammalian subject, the method comprising administering the conjugate of claim 14 to the mammalian subject in an amount effective to enhance the protective immune response to Streptococcus pneumoniae in the mammalian subject.


SEQUENCE LISTING (3 sequences)

SEQ ID NO: 1 - protein, 12 aa, Salmonella typhimurium
GAVQNRFNSAIT

SEQ ID NO: 2 - DNA, 2328 bp, Artificial sequence
  Synthetic nucleic acid molecule
atggaagaatctccacaagttgtcgaaaaatcttcattagagaagaaatatgaggaagcaaaagcaaaagctgatactgccaagaaagattacgaaacggctaaaaagaaagcagaagacgctcagaaaaagtatgaagatgatcagaagagaactgaggagaaagctcgaaaagaagcagaagcatctcaaaaattgaatgatgtggcgcttgttgttcaaaatgcatataaagagtaccgagaagttcaaaatcaacgtagtaaatataaatctgacgctgaatatcagaaaaaattaacagaggtcgactctaaaatagagaaggctaggaaagagcaacaggacttgcaaaataaatttaatgaagtaagagcagttgtagttcctgaaccaaatgcgttggctgagactaagaaaaaagcagaagaagctaaagcagaagaaaaagtagctaagagaaaatatgattatgcaactctaaaggtagcgctagcgaagaaagaagtagaggctaaggaacttgaaattgaaaaacttcaatatgaaatttctactttggaacaagaagttgctactgctcaacatcaagtagataatttgaaaaaacttcttgctggtgcggatcctgatgatggcacagaagttatagaagctaaattaaaaaaaggagaagctgagctaaacgctaaacaagctgagttagcaaaaaaacaaacagaacttgaaaaacttcttgacggccttgatcctgaaggtaagactcaggatgaattagataaaaagcttgcacaagtcattaatacaaacagcctgtcgctgttgacccagaataacctgaacaaatctcagtcctcactgagttccgctattgagcgtctgtcctctggtctgcgtatcaacagcgcgaaagacgatgcggcaggccaggcgattgctaaccgcttcacttctaatatcaaaggtctgactcaggcttcccgtaacgctaacgacggcatttctattgcgcagaccactgaaggtgcgctgaatgaaatcaacaacaacctgcagcgtgtgcgtgagttgtctgttcaggccactaacgggactaactctgattccgatctgaaatctatccaggatgaaattcagcaacgtctggaagaaatcgatcgcgtttctaatcagactcaatttaacggtgttaaagtcctgtctcaggacaaccagatgaaaatccaggttggtgctaacgatggtgaaaccattaccatcgatctgcaaaaaattgatgtgaaaagccttggccttgatgggttcaatgttaatgggccaaaagaagcgacagtgggtgatctgaaatccagcttcaagaatgttacgggttacgacacctatgcagcgggtgccgataaatatcgtgtagatattaattccggtgctgtagtgactgatgcagcagcaccggataaagtatatgtaaatgcagcaaacggtcagttaacaactgacgatgcggaaaataacactgcggttgatctctttaagaccactaaatctactgctggtaccgctgaagccaaagcgatagctggtgccattaaaggtggtaaggaaggagatacctttgattataaaggcgtgacttttactattgatacaaaaactggtgatgacggtaatggtaaggtttctactaccatcaatggtgaaaaagttacgttaactgtcgctgatattgccactggcgcgacggatgttaatgctgctaccttacaatcaagcaaaaatgtttatacatctgtagtgaacggtcagtttacttttgatgataaaaccaaaaacgagagtgcgaaactttctgatttggaagcaaacaatgctgttaagggcgaaagtaaaattacagtaaatggggctgaatatactgctaacgccacgggtgataagatcaccttagctggcaaaaccatgtttattgataaaacagcttctggcgtaagtacattaatcaatgaagacgctgccgcagccaagaaaagtaccgctaacccactggcttcaattgattctgcattgtcaaaagtggacgcagttcgttcttctctgggggcaattcaaaaccgttttgattcagccattaccaaccttggcaatacggtaaccaatctgaactccgcgcgtagccgtatcgaagatgctgactatgcaacggaagtttctaatatgtctaaagcgcagattctgcagcaggctggtacttccgttctggcgcaggctaaccaggttccgcaaaacgtcctctctttactgcgtctcgagcaccaccaccaccaccactga

SEQ ID NO: 3 - protein, 775 aa, Artificial sequence
  Synthetic polypeptide
MEESPQVVEKSSLEKKYEEAKAKADTAKKDYETAKKKAEDAQKKYEDDQKRTEEKARKEAEASQKLNDVALVVQNAYKEYREVQNQRSKYKSDAEYQKKLTEVDSKIEKARKEQQDLQNKFNEVRAVVVPEPNALAETKKKAEEAKAEEKVAKRKYDYATLKVALAKKEVEAKELEIEKLQYEISTLEQEVATAQHQVDNLKKLLAGADPDDGTEVIEAKLKKGEAELNAKQAELAKKQTELEKLLDGLDPEGKTQDELDKKLAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNGPKEATVGDLKSSFKNVTGYDTYAAGADKYRVDINSGAVVTDAAAPDKVYVNAANGQLTTDDAENNTAVDLFKTTKSTAGTAEAKAIAGAIKGGKEGDTFDYKGVTFTIDTKTGDDGNGKVSTTINGEKVTLTVADIATGATDVNAATLQSSKNVYTSVVNGQFTFDDKTKNESAKLSDLEANNAVKGESKITVNGAEYTANATGDKITLAGKTMFIDKTASGVSTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLRLEHHHHHH